(19) United States
(12) Patent Application Publication
(10) Pub. No.: US 2025/0145555 A1
(43) Pub. Date: May 8, 2025

(54) PROCESSES FOR SEPARATING ORGANIC PRODUCTS FROM ORGANIC RECYCLE STREAMS

(71) Applicant: Westlake Epoxy Inc., Houston, TX (US)

(72) Inventors: Christin BARRON, Dusseldorf (DE); Claudia REMUS, Rheinberg (DE); Gunda KUHLMANN, Hamm (DE); Gabriele BADINI, Dusseldorf (DE)

(73) Assignee: Westlake Epoxy Inc., Houston, TX (US)

(21) Appl. No.: 18/387,839

(22) Filed: Nov. 7, 2023

(51) Int. Cl. C07C 37/72; C08G 8/10

(52) U.S. Cl. CPC C07C 37/72; C08G 8/10

(57) ABSTRACT

Embodiments of the present disclosure generally relate to processes for separating organic products from organic recycle streams. The processes can be utilized to form fractions that can be used for further processing. In some embodiments, one or more of the fractions can have a higher concentration of phenol than the concentration of phenol in the organic recycle stream that is separated. In some embodiments, a fraction can include phenolic compounds, a glycidation substrate, a curing agent, an accelerator, or combinations thereof. In some embodiments, a fraction can include phenolic oligomers. The fractions separated can be used to form, for example, a phenol-formaldehyde resin.

### FIELD

Embodiments of the present disclosure generally relate to processes for separating organic products from organic recycle streams.

### BACKGROUND

Fiber-reinforced thermoset composites, such as epoxy resin and phenolic resin composites, are widely used as structural, light weight components. After use, such composites are mainly landfilled as they are viewed to be non-recyclable. New recycling technologies have begun to address this issue. Here, methods such as pyrolysis, solvolysis, thermolysis, and catalytic depolymerization are used to separate organic recycle streams (as oils) from the fibers. These organic recycle streams includes valuable products such as phenolic compounds, resins, and other organic compounds. However, due to the complexity and high expense of isolating the valuable organic products from the bulk organic recycle streams, the organic recycle streams are largely incinerated or used as fuel.

Therefore, there is a need for new and improved processes for separating organic products from organic recycle streams. There is also a need for new and improved processes for producing a composition enriched in phenol from organic recycle streams.

### SUMMARY

Embodiments of the present disclosure generally relate to processes for separating organic products from organic recycle streams, and more specifically to processes for producing a composition enriched in phenol from organic recycle streams. Unlike conventional technologies, embodiments described herein can enable removal of phenol fractions and other organic fractions from resin recycle streams and resin waste streams. The phenol fractions and other organic fractions can be utilized to produce, for example, novolak resins and epoxy resins, among other materials.

In an embodiment is provided a process that includes (a) contacting a feedstock with a first organic solvent and water and forming a first aqueous stream comprising organic acids and a first organic stream comprising phenol, phenolic compounds, and phenolic oligomers, the feedstock having a first concentration of the phenol; (b) contacting the first organic stream with an aqueous base and forming a second aqueous stream comprising the phenol and the phenolic oligomers and a second organic stream comprising the phenolic compounds; (c) contacting the second aqueous stream with an aqueous acid and forming a precipitate comprising the phenolic oligomers and a third aqueous stream comprising the phenol; and (d) contacting the third aqueous stream with a second organic solvent and forming a composition having a second concentration of the phenol that is greater than the first concentration of the phenol.

In another embodiment is provided a process that includes contacting a feedstock comprising thermochemically derived products of an epoxy-resin composite material, catalytically depolymerized products of an epoxy-resin composite material, or combinations thereof with an aprotic solvent and water and forming a first aqueous stream comprising organic acids and a first organic stream comprising phenol, phenolic compounds, and phenolic oligomers, the feedstock having a first concentration of the phenol. The process further includes contacting the first organic stream with an aqueous base and forming a second aqueous stream comprising the phenol and the phenolic oligomers and a second organic stream comprising the phenolic compounds, wherein: the second organic stream further comprises a glycidation substrate, a curing agent, an accelerator, or combinations thereof; the aqueous base comprises an alkali metal hydroxide; and the second organic stream comprises a lower concentration of the phenol than the second aqueous stream.

In an embodiment is provided a process that includes (a) contacting a feedstock comprising products from solvolysis of an epoxy-resin composite material, pyrolysis of an epoxy-resin composite material, thermolysis of an epoxy-resin composite material, catalytic depolymerization of an epoxy-resin composite material, or combinations thereof with a first organic solvent and water and forming a first aqueous stream comprising organic acids and a first organic stream comprising phenol, phenolic compounds, and phenolic oligomers, the feedstock having a first concentration of the phenol. The process further includes (b) contacting the first organic stream with an aqueous base and forming a second aqueous stream comprising the phenol and the phenolic oligomers and a second organic stream comprising the phenolic compounds. The process further includes (c) contacting the second aqueous stream with an aqueous acid and forming a precipitate comprising the phenolic oligomers and a third aqueous stream comprising the phenol. The process further includes (d) contacting the third aqueous stream with a second organic solvent and forming a composition having a second concentration of the phenol that is greater than the first concentration of the phenol.

In another embodiment is provided a process that includes (a) contacting a feedstock comprising thermochemically derived products of an epoxy-resin composite material, catalytically depolymerized products of an epoxy-resin composite material, or combinations thereof with a first aprotic solvent and water and forming a first aqueous stream comprising organic acids and a first organic stream comprising phenol, phenolic compounds, and phenolic oligomers, the feedstock having a first concentration of the phenol. The process further includes (b) contacting the first organic stream with an aqueous base and forming a second aqueous stream comprising the phenol and the phenolic oligomers and a second organic stream comprising the phenolic compounds, the aqueous base comprising an alkali metal hydroxide, the second organic stream comprising a lower concentration of the phenol than the second aqueous stream. The process further includes (c) contacting the second aqueous stream with an aqueous inorganic acid and forming a precipitate comprising the phenolic oligomers and a third aqueous stream comprising the phenol. The process further includes (d) contacting the third aqueous stream with a second aprotic solvent and forming a composition having a second concentration of the phenol that is greater than the first concentration of the phenol.

In another embodiment, a process for forming a phenol-formaldehyde resin is provided. The process includes contacting a feedstock comprising products from solvolysis of an epoxy-resin composite material, pyrolysis of an epoxy-resin composite material, thermolysis of an epoxy-resin composite material, catalytic depolymerization of an epoxy-resin composite material, or combinations thereof with a first organic solvent and water and forming a first aqueous stream comprising organic acids and a first organic stream comprising phenol, phenolic compounds, and phenolic oligomers, the feedstock having a first concentration of the phenol. The process further includes contacting the first organic stream with an aqueous base and forming a second aqueous stream comprising the phenol and the phenolic oligomers and a second organic stream comprising the phenolic compounds. The process further includes contacting the second aqueous stream with an aqueous acid and forming a precipitate comprising the phenolic oligomers and a third aqueous stream comprising the phenol. The process further includes contacting the third aqueous stream with a second organic solvent and forming a composition having a second concentration of the phenol that is greater than the first concentration of the phenol. The process further includes converting the composition enriched in the phenol to a phenol-formaldehyde resin.

In another embodiment, a process for producing a composition enriched in phenol is provided. The process includes (a) contacting a feedstock comprising products from solvolysis of an epoxy-resin composite material, pyrolysis of an epoxy-resin composite material, thermolysis of an epoxy-resin composite material, catalytic depolymerization of an epoxy-resin composite material, or combinations thereof with a first organic solvent and water to obtain a first aqueous stream comprising organic acids and a first organic stream comprising phenol, phenolic compounds, and phenolic oligomers. The process further includes (b) contacting the first organic stream with an aqueous base to form a second aqueous stream comprising the phenol and a second organic stream comprising the phenolic compounds. The process further includes (c) contacting the second aqueous stream with an aqueous acid to form a precipitate comprising the phenolic oligomers and a third aqueous stream comprising the phenol; and (d) contacting the third aqueous stream with a second organic solvent to form a composition enriched in the phenol.

In another embodiment, a process for producing a composition enriched in phenol is provided. The process includes (a) contacting a feedstock comprising thermochemically derived products of an epoxy-resin composite material, catalytically depolymerized products of an epoxy-resin composite material, or combinations thereof with a first aprotic solvent and water to obtain a first aqueous stream comprising organic acids and a first organic stream comprising phenol. The process further includes (b) contacting the first organic stream with an aqueous base to form a second aqueous stream comprising the phenol and a second organic stream comprising phenolic compounds, the aqueous base comprising an alkali metal hydroxide, the second organic stream comprising a lower amount of phenol than the second aqueous stream. The process further includes (c) contacting the second aqueous stream with an aqueous inorganic acid to form a precipitate comprising phenolic oligomers and a third aqueous stream comprising the phenol; and (d) contacting the third aqueous stream with a second aprotic solvent to form a composition enriched in the phenol.

In another embodiment, a process for forming a phenol-formaldehyde resin is provided. The process includes contacting a feedstock comprising products from solvolysis of an epoxy-resin composite material, pyrolysis of an epoxy-resin composite material, thermolysis of an epoxy-resin composite material, catalytic depolymerization of an epoxy-resin composite material, or combinations thereof with a first organic solvent and water to obtain a first aqueous stream comprising organic acids and a first organic stream comprising phenol, phenolic compounds, and phenolic oligomers. The process further includes contacting the first organic stream with an aqueous base to form a second aqueous stream comprising the phenol and a second organic stream comprising phenolic compounds; contacting the second aqueous stream with an aqueous acid to form a precipitate comprising phenolic oligomers and a third aqueous stream comprising the phenol; and contacting the third aqueous stream with a second organic solvent to form a composition enriched in the phenol. The process further includes converting the composition enriched in phenol to a phenol-formaldehyde resin.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1A is a flowchart showing selected operations of a process for separating products from organic recycle streams according to at least one embodiment of the present disclosure.

FIG. 1B is a flow diagram illustrating a process for separating products from organic recycle streams according to at least one embodiment of the present disclosure.

FIG. 2 shows an overlay of infrared (IR) spectra of the organic, oligomeric, and phenolic fractions of separated pyrolysis oil according to at least one embodiment of the present disclosure.

FIG. 3 shows an overlay of chromatograms of crude pyrolysis oil and separated fractions as measured by gel permeation chromatography (GPC) according to at least one embodiment of the present disclosure.

FIG. 4 shows an overlay of chromatograms of novolak resins produced from 20% to 100% recycled material (phenolic fraction) as measured by ultra-high-performance liquid chromatography (UHPLC) according to at least one embodiment of the present disclosure.

Figures included herein illustrate various embodiments of the disclosure. It is contemplated that elements and features of one embodiment may be beneficially incorporated in other embodiments without further recitation.

### DETAILED DESCRIPTION

Embodiments of the present disclosure generally relate to processes for separating organic products from organic recycle streams. The composition enriched in phenol can be utilized to form, for example, novolak resins. Embodiments of the present disclosure can also be utilized to form fractions or streams containing phenol, phenolic compounds, phenolic oligomers, or combinations thereof which can be utilized to form, for example, novolak resins. Embodiments of the present disclosure can also be utilized to form fractions or streams containing curing agents, accelerators, glycidation substrates, advancement resins, or combinations thereof. Such curing agents, accelerators, glycidation substrates, advancement resins, or combinations thereof can then be used to form, for example, epoxy resins.

As described above, conventional technologies have been unable to address the re-use or recyclability of thermosets present in fiber-reinforced thermoset composites such as epoxy resin composites and other resin composites. The conventional wisdom has been to view such thermosets as non-recyclable. In contrast, processes described herein can enable sustainable recycling technologies for fiber-reinforced thermoset composites such as epoxy resin composites and other resin composites. Here, processes of the present disclosure can enable retrieval of valuable products such as phenol, phenolic compounds, and other organic compounds from solvolyzed fiber-reinforced thermoset composites (solvolysis oil), pyrolyzed fiber-reinforced thermoset composites (pyrolysis oil), thermolyzed fiber-reinforced thermoset composites (thermolysis oil), catalytically decomposed fiber-reinforced thermoset composites, or any other suitable recycling process. As a consequence, processes described herein can enable sustainable recycling technologies for fiber-reinforced thermoset composites such as epoxy resin composites and other resin composites. In addition, processes described herein can also mitigate landfilling issues related to fiber-reinforced thermoset composites. Further, processes described herein can enable circularity such that fiber-reinforced thermoset composites, such as epoxy resin composites and other resin composites, can be used as a resource rather than left as a waste.

The use of headings is for purposes of convenience only and does not limit the scope of the present disclosure. Embodiments described herein can be combined with other embodiments.

As used herein, a “composition” can include component(s) of the composition, reaction product(s) of two or more components of the composition, a remainder balance of remaining starting component(s), or combinations thereof. Compositions of the present disclosure can be prepared by any suitable mixing process.

As used herein, “a composition enriched in phenol” means that the relative amount (or concentration) of phenol in a composition after separation is greater than the relative amount (or concentration) of phenol in a feedstock before the separation. For example, if a feedstock includes 1% phenol before separation, the composition formed after the separation would include greater than 1% phenol.

As used herein, “phenol” refers to the compound of formula (I):

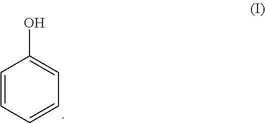

As used herein, “recycled phenol” refers to the phenol (compound of formula (I)) present in a composition enriched in phenol. Recycled phenol can also be present in other fractions formed by processes described herein.

As used herein, “phenolic compounds” include those compounds represented by formula (II).

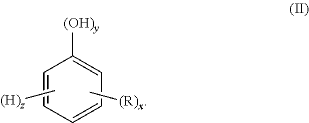

The phenolic compound of formula (II) includes at least one hydrogen atom (H), such that z is at least one. In formula (II), R is a group substituted for a hydrogen atom on the aromatic ring, and OH is a hydroxyl substituted for a hydrogen atom on the aromatic ring.

In some embodiments, z is from 1 to 4, such as 1, 2, 3, or 4; x is from 1 to 4 (such as 1, 2, 3, or 4); y is from 1 to 4 (such as 1, 2, 3, or 4); and combinations thereof. A total of x, y, and z on the aromatic ring of formula (II) is 6. When x is more than 1, each R group can be the same or different.

Each R group of formula (II) can be an unsubstituted hydrocarbyl, a substituted hydrocarbyl, or a functional group comprising at least one element from Group 13-17 of the periodic table of the elements. When an R group is a functional group comprising at least one element from Group 13-17, the R group can be halogen (F, Cl, Br, or I), O, N, Se, Te, P, As, Sb, S, B, Si, Ge, Sn, Pb, and the like, such as C (O) R*, C (C) NR*2, C (O) OR*, NR*2, OR*, SeR*, TeR*, PR*2, AsR*₂, SbR*₂, SR*, SOₓ (where x=2 or 3), BR*₂, SiR*₃, GeR*₃, SnR*₃, PbR*₃, and the like, where R* is, independently, hydrogen or unsubstituted hydrocarbyl, or where at least one heteroatom has been inserted within the unsubstituted hydrocarbyl.

Each R group of formula (II) can have, independently, any suitable number of carbon atoms such as from 1 to 20 carbon atoms, such as from 1 to 10 carbon atoms, such as from 1 to 5 carbon atoms, such as from 1 to 4 carbon atoms. In some embodiments, the number of carbon atoms in each R group of formula (II) can be, independently, 1, 2, 3, 4, 5, 6, 7, 8, 9, 10, 11, 12, 13, 14, 15, 16, 17, 18, 19, or 20. Each of the foregoing numbers can be preceded by the word “about,” “at least about,” “less than about,” or “more than about,” and any of the foregoing numbers can be used singly to describe an open-ended range or in combination to describe a close-ended range. Each R group of formula (II) can be, independently, linear or branched, saturated or unsaturated, cyclic or acyclic, aromatic or not aromatic. Regarding saturation, each R group of formula (II) can be, independently, fully saturated, partially unsaturated, or fully unsaturated.

In some examples, one or more R groups of formula (II) can be an unsubstituted hydrocarbyl. An “unsubstituted hydrocarbyl” refers to a group that consists of hydrogen and carbon atoms only. Illustrative, but non-limiting, examples of unsubstituted hydrocarbyl include an alkyl group having from 1 to 20 carbon atoms such as methyl, ethyl, n-propyl, isopropyl, n-butyl, iso-butyl, sec-butyl, and tert-butyl, pentyl, hexyl, heptyl, octyl, ethyl-2-hexyl, isooctyl, nonyl, n-decyl, isodecyl, or isomers thereof; a cycloaliphatic group having from 3 to 20 carbon atoms such as, for example, cyclopentyl or cyclohexyl; an aromatic group having from 6 to 20 carbon atoms such as, for example, phenyl or naphthyl; or any combination thereof. In some embodiments, one or more R groups of formula (II) can be a linear or branched alkenyl having from 1 to 20 carbon atoms, such as from 3 to 10 carbon atoms. The term “alkenyl” refers to a hydrocarbyl having at least one double bond. An illustrative, but non-limiting, example of alkenyl includes allyl (for example, —CH₂CH—CH₂).

In some embodiments, one or more R groups of formula (II) can be a substituted hydrocarbyl. A “substituted hydrocarbyl” refers to an unsubstituted hydrocarbyl in which at least one hydrogen of the unsubstituted hydrocarbyl has been substituted with at least one heteroatom or heteroatom-containing group, such as one or more elements from Group 13-17 of the periodic table of the elements, such as halogen (F, Cl, Br, or I), O, N, Se, Te, P, As, Sb, S, B, Si, Ge, Sn, Pb, and the like, such as C (O) R*, C (C) NR*₂, C (O) OR*, NR*₂, OR*, SeR*, TeR*, PR*₂, AsR*₂, SbR*₂, SR*, SOₓ (where x=2 or 3), BR*₂, SiR*₃, GeR*₃, SnR*₃, PbR*₃, and the like, where R* is, independently, hydrogen or unsubstituted hydrocarbyl, or where at least one heteroatom has been inserted within the unsubstituted hydrocarbyl.

Illustrative, but non-limiting, examples of substituted hydrocarbyls include —OR*, where the oxygen atom is connected to the ring and where R* can include 1 to 10 carbon atoms, such as 1 to 5 carbon atoms, such as 2 to 4 carbon atoms. For example, —OR* can be alkyloxy such as methoxy, ethoxy, propoxy, butoxy, and isomers thereof.

In some embodiments, each R group of formula (II) is a group that is non-reactive with an epoxide-bearing reactant. For example, and in at least one embodiment, each R group of formula (II) is, independently, -(C1-C5) alkyl or —O (C1-C5) alkyl.

In at least one embodiment, the phenolic compound of formula (II) is a monophenol (y is 1).

In formula (II), and in some embodiments, at least one R group is located at an ortho position on the aromatic ring relative to the hydroxyl group. In certain embodiments, an R group of formula (II) is located at each ortho position on the ring relative to the hydroxyl group.

As used herein, “phenolic oligomers” refers to oligomers of phenol (compound of formula (I)), oligomers of phenolic compounds represented by formula (II), or combinations thereof.

### Separation Processes

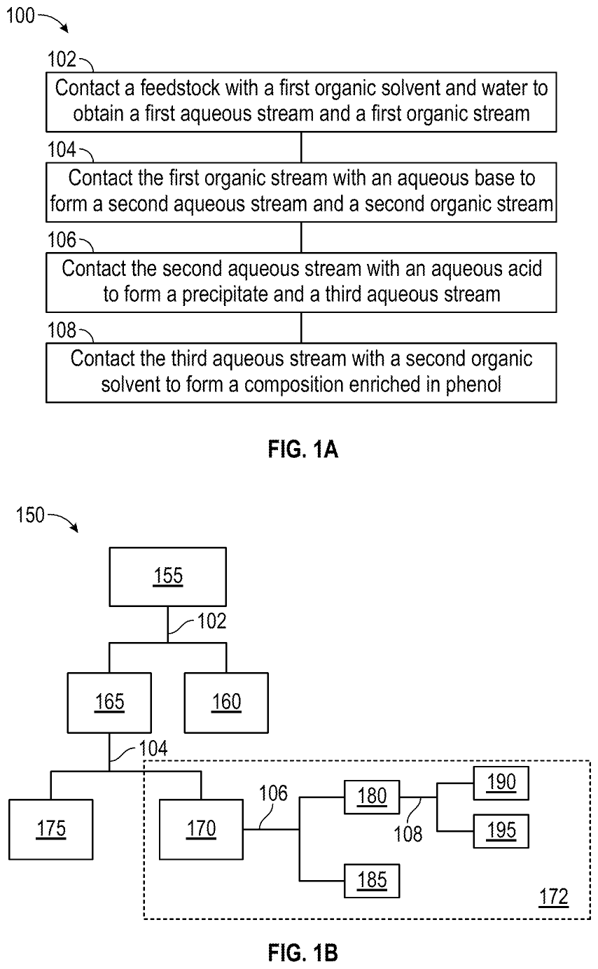

FIG. 1A is a flowchart showing selected operations of a process 100 for separating organic products from an organic recycle stream according to at least one embodiment of the present disclosure. The process 100 produces a composition enriched in phenol. The composition enriched in phenol has a higher concentration of phenol than a concentration of phenol in the feed organic recycle stream. The phenol obtained from the processes is a recycled phenol. FIG. 1B is a flow diagram 150 illustrating an embodiment of the process 100 for producing a composition enriched in phenol according to at least one embodiment of the present disclosure.

During process 100, suitable solvents can be used during various operations. Suitable solvents used during one or more operations of process 100 include, but are not limited to, aqueous solvents, organic solvents, or combinations thereof. Aqueous solvents can be selected from the group consisting of water, distilled water, deionized water, ultra-pure water, and combinations thereof. Organic solvents can be selected from the group consisting of halogenated solvents, alcohol solvents, alkylcarbonate solvents, ketone solvents, hydrocarbon solvents, ester solvents, ether solvents, and combinations thereof. Halogenated solvents can be selected from the group consisting of dichloromethane, chloroform, and combinations thereof. Alcohol solvents can be selected from the group consisting of ethanol (EtOH), methanol, isopropanol, n-propanol, n-butanol, isobutanol, sec-butanol, an amyl alcohol (such as n-pentanol, isopentanol, and sec-pentanol), and combinations thereof. Alkylcarbonate solvents can be selected from the group consisting of dimethyl carbonate, diethyl carbonate, dipropyl carbonate, and combinations thereof. Ketone solvents can include acetone. Hydrocarbon solvents can be selected from the group consisting of hexane, pentane, cyclohexane, benzene, toluene, and combinations thereof. Ester solvents can include ethyl acetate. Ether solvents can be selected from the group consisting of dimethyl ether, diethyl ether, tetrahydrofuran, dipropylene glycol dimethyl ether, methyl tert-butyl ether, glycol ether, and combinations. Other solvents such as ethyl acetate, dimethylformamide, acetonitrile, N-methyl-2-pyrrolidone, dimethyl sulfoxide, or combinations thereof can be utilized. Mixtures of solvents can be utilized.

Process 100 begins with contacting a feedstock 155 with a first organic solvent and water to form a first aqueous stream 160 and a first organic stream 165 at operation 102. The feedstock 155 utilized for operation 102 comprises, consists essentially of, or consists of an organic recycle stream. The feedstock has a first concentration of phenol.

The organic recycle stream can include epoxy resins, phenolic resins, or combinations thereof. Additionally, or alternatively, the organic recycle stream can include those streams used or made during processing or manufacturing of resins, those streams used or made during processing or manufacturing thermosets, those streams used or made during processing or manufacturing fiber-reinforced thermoset composites, or combinations thereof.

Additionally, or alternatively, the organic recycle stream can include materials sourced or derived from fiber-reinforced thermoset composites. As described above, fiber-reinforced thermoset composites can be subjected a thermochemical process, such as pyrolysis, solvolysis, thermolysis, catalytic depolymerization, or combinations thereof, to separate the organic recycle stream from the fibers. These organic recycle streams can be products, for example, oils such as pyrolysis oils, solvolysis oils, thermolysis oils, oils from catalytic depolymerization, or combinations thereof, among others. Such oils contain valuable organic compounds such as phenolic compounds, resins, and other organic compounds.

Accordingly, and in some embodiments, the organic recycle stream can include products from solvolysis of an epoxy-resin composite material, products from solvolysis of a phenolic-resin composite material, products from pyrolysis of an epoxy-resin composite material, products from pyrolysis of a phenolic-resin composite material, products from thermolysis of an epoxy-resin composite material, products from thermolysis of a phenolic-resin composite material, products from catalytic depolymerization of an epoxy-resin composite material, products from thermolysis of a phenolic-resin composite material, or combinations thereof, as well as products produced from any suitable recycling process of composite materials. Such products can be in the form of oils. Thermolysis is typically performed by heating a composite material to form gaseous, liquid, and solid products. Catalytic depolymerization can involve use of transition metal catalysts, such as ruthenium-based catalysts, to disconnect bonds (such as C (alkyl)-O bonds) in the composite materials, thereby forming products.

During solvolysis of epoxy-resin composites, a solvent and acid can be utilized to separate a solvolysis oil from the fibers. Here, solvents as acetone, water, ethanol, 2-propanol, or supercritical solvent mixtures can be used. Acids such as nitric acid, acetic acid, p-toluene sulfonic acid (p-TsOH), or combinations thereof can be utilized s. Products from solvolysis heavily depend on the recycling process used and are not intended to be limited by the description herein.

Illustrative, but non-limiting examples of products (a solvolysis oil) from solvolysis of epoxy-resin composites can include, but are not limited to: phenol; aniline; quinoline; 4-(1-methylethyl) phenol; 4-ethylphenol; phenol, 3-(1-methylethyl)-; 2H-1-Benzopyran, 3,4-dihydro-; 3-phenoxy-1,2-propanediol; p-hydroxybiphenyl; 2-propanol, 1-phenoxy-3-(phenylamino)-; 1H-indole, 2,5-dimethyl; or combinations thereof. The remaining amount of the solvolysis oil can include one or more of those compounds described below for the pyrolysis oil among other compounds. Additionally, or alternatively, other products of the solvolysis present in the solvolysis oil can include phenolic compounds, phenol oligomers glycidation substrates, curing agents, accelerators, advancement resins, amine derivatives, phenyl amine derivatives, or combinations thereof can also be present in the solvolysis oil.

In some embodiments, the solvolysis oil can include phenol, phenolic compounds, and phenol oligomers in an amount of about 2 wt % to about 25 wt %, such as from about 10 wt % to about 17 wt %, based on a total weight percent of the solvolysis oil, though other amounts are contemplated. The total weight of solvolysis oil does not exceed 100 wt %. The remaining amount of the solvolysis oil can include one or more of those compounds described above for the solvolysis oil among other compounds.

During pyrolysis of epoxy-resin composites, the composites are heated to about 350° C. to about 500° C. in the absence or in the presence of oxygen to form an organic fraction (pyrolysis oil).

Illustrative, but non-limiting, examples of products from pyrolysis of epoxy-resin composites (a pyrolysis oil) can include, but are not limited to: phenol; p-cumenol; 3-isopropylphenol; 4,4′-(1-methylethylidene) bis-phenol; toluene; o-cresol; 4-ethylphenol; p-cresol; 4-isopropyl-3-methylphenol; 2-ethylphenol; p-isopropenylphenol; 2-methyl-2-(4′-hydroxyphenyl) pentanone-4; acetone; aniline; m-cresol; benzene; ethylbenzene; styrene; xylene; or combinations thereof. In some embodiments, phenol, phenolic compounds, and phenol oligomers in an amount of about 2 wt % to about 30 wt %, such as from about 10 wt % to about 22 wt %, based on a total weight percent of the pyrolysis oil, though other amounts are contemplated. The total weight of pyrolysis oil does not exceed 100 wt %. The remaining amount of the pyrolysis oil can include one or more of those compounds described above for the solvolysis oil among other compounds. Additionally, or alternatively, other products of the pyrolysis present in the pyrolysis oil can include phenolic compounds, phenol oligomers glycidation substrates, curing agents, accelerators, advancement resins, amine derivatives, phenyl amine derivatives, or combinations thereof can also be present in the pyrolysis oil.

Each of the aforementioned solvolysis oil, pyrolysis oil, thermolysis oil, oils from catalytic decomposition, components thereof, or combinations thereof can make up at least a portion of the feedstock 155 for processes described herein.

The water utilized for operation 102 can comprise, consist essentially of, or consist of tap water, distilled water, deionized water, ultra-pure water, or combinations thereof. In some embodiments the water can include salts. A weight ratio of feedstock to water used for operation 102 can be from about 1:50 to about 50:1, such as from about 1:35 to about 35:1, such as from about 1:20 to about 20:1, such as from about 1:15 to about 15:1, such as from about 1:10 to about 10:1, such as from about 5:1 to about 1:5, such as from about 3:1 to about 1:3, such as from about 2:1 to about 1:2, such as from about 1.2:1 to about 1:1.2, such as about 1:1, though other amounts are contemplated. Any of the foregoing numbers can be used singly to describe an open-ended range or in combination to describe a close-ended range.

The first organic solvent utilized for operation 102 can include those organic solvents described above. In some embodiments, the first organic solvent utilized for operation 102 can comprise, consist essentially of, or consist of halogenated solvent, ether solvent, ketone solvent, ester solvent, or combinations thereof. In at least one embodiment, the first organic solvent utilized for operation 102 is selected from the group consisting of halogenated solvent, ether solvent, ketone solvent, ester solvent, and combinations thereof. In some embodiments, the first organic solvent comprises an aprotic solvent. Aprotic solvents are solvents that do not donate protons (H*) to solution.

In some embodiments, the first organic solvent utilized for operation 102 can comprise, consist essentially of, or consist of dichloromethane, chloroform, methyl tert-butyl ether, ethyl acetate, toluene, benzene, hexane, cyclohexane, and combinations thereof. In at least one embodiment, the first organic solvent utilized for operation 102 is selected from the group consisting of dichloromethane, chloroform, methyl tert-butyl ether, ethyl acetate, toluene, benzene, hexane, cyclohexane, or combinations thereof.

A weight ratio of the feedstock 155 to first organic solvent used for operation 102 can be from about 1:50 to about 50:1, such as from about 1:35 to about 35:1, such as from about 1:20 to about 20:1, such as from about 1:15 to about 15:1, such as from about 1:10 to about 10:1, such as from about 5:1 to about 1:5, such as from about 3:1 to about 1:3, such as from about 2:1 to about 1:2, such as from about 1.2:1 to about 1:1.2, such as about 1:1, though other amounts are contemplated. Any of the foregoing numbers can be used singly to describe an open-ended range or in combination to describe a close-ended range.

During operation 102, the feedstock 155, water, and first organic solvent can be charged to a vessel. The resulting mixture can be mixed, stirred, or otherwise agitated under mixing conditions effective to form the first aqueous stream 160 and the first organic stream 165. Mixing conditions of operation 102 can include a temperature of about 15° C. to about 30° C. and an ambient pressure that is about 1 atm. Elevated temperatures can be used if desired. However, if elevated temperatures are used during mixing, the mixing temperature should be below the temperature at which the water and the first organic solvent boils.

Mixing conditions of operation 102 can include stirring, mixing, agitating, or combinations thereof by using suitable devices. Suitable devices can include a mechanical stirrer such as an overhead stirrer, a magnetic stirrer (for example, placing a magnetic stir bar in the vessel above a magnetic stirrer), or other suitable devices. For example, a stirrer having a blade or propeller can be rotated by receiving rotational power from a stirring motor to stir the components at suitable rotation speeds, such as from about 50 revolutions per minute (rpm) to about 1,500 rpm, such as from about 75 rpm to about 1,000 rpm, such as from about 100 rpm to about 900 rpm, such as from about 200 rpm to about 800 rpm, such as from about 300 rpm to about 700 rpm, such as from about 400 rpm to about 600 rpm, such as from about 450 rpm to about 550 rpm, such as about 500 rpm. Any of the foregoing numbers can be used singly to describe an open-ended range or in combination to describe a close-ended range. Other rotation speeds are contemplated and can be selected based on the ability to mix the components sufficiently. Mixing conditions of operation 102 can include utilizing a non-reactive gas, such as N₂, Ar, or combinations thereof. For example, a non-reactive gas can be introduced to the feedstock 155, water, and first organic solvent to degas various components or otherwise remove unwanted gases such as oxygen from the mixture.

Mixing, stirring, or agitating during operation 102 can be performed for any suitable period, such as from about 1 min to about 48 h, such as from about 5 min to about 24 h, such as from about 30 min to about 10 h, such as from about 1 h to about 5 h, such as from about 2 h to about 3 h, though other periods are contemplated. Any of the foregoing numbers can be used singly to describe an open-ended range or in combination to describe a close-ended range.

After mixing, the mixture of feedstock 155, water, and solvent is separated by suitable liquid-liquid separation techniques, such as decanting, distillation, extraction, extractive distillation, or combinations thereof, among other techniques. After separation, the first aqueous stream 160 and the first organic stream 165 is obtained. The first aqueous stream 160 can include organic acids such as p-toluene sulfonic acid or hexahydrophthalic acid, among other acids. Any suitable organic acid may be present depending on, for example, the acid used to form the feedstock.

The first organic stream 165 can include phenolic compounds (including phenol) among other organic compounds such as one or more of those compounds described above. In some embodiments, the first aqueous stream has a higher amount of organic acids (for example, p-toluene sulfonic acid or hexahydrophthalic acid) than the amount of organic acids in the first organic stream.

If desired, the first aqueous stream 160 can be subjected to operation 102, by which the first aqueous stream 160 can be contacted with the first organic solvent and the resulting mixture separated. After separation, the organic layer can be combined with the first organic stream 165. Subjecting the first aqueous stream 160 to operation 102 can be utilized to retrieve any remaining phenol, other organic compounds, or combinations thereof in the first aqueous stream 160. Operation 102 can be performed any suitable number of times such as 1, 2, 3, 4, or more times. Each of the organic layers can be retrieved and combined to form the first organic stream 165.

In some embodiments, and prior to proceeding with further operations of process 100, solvent such as organic solvent or water can be removed from the first aqueous stream 160, the first organic stream 165, or both. Solvent removal can be accomplished by suitable techniques such as distillation, vacuum distillation, or combinations thereof.

Process 100 further includes contacting the first organic stream 165 with an aqueous base to form a second aqueous stream 170 and a second organic stream 175 at operation 104.

The aqueous base utilized for operation 104 can comprise, consist essentially of, or consist of an alkali metal hydroxide. Suitable alkali metal hydroxides can include lithium hydroxide (LiOH), sodium hydroxide (NaOH), potassium hydroxide (KOH), rubidium hydroxide (RbOH), cesium hydroxide (CsOH), or combinations thereof, such as LiOH, NaOH, KOH, or combinations thereof. In some embodiments, the alkali metal hydroxide is selected from the group consisting of LiOH, NaOH, KOH, and combinations thereof.

The aqueous base can be added to the first organic stream 165 until the pH reaches a desired value and maintains that value such that phenol and phenolic compounds are deprotonated, converting the phenol and phenolic compounds to their corresponding phenolates. Here, and in some embodiments, operation 104 can include adding the aqueous base to the first organic stream 165 to form a mixture having a desired pH value. By converting phenol and phenolic compounds to their corresponding phenolates, the compounds become water soluble, allowing for transfer of phenol and phenolic compounds to the second aqueous stream 170.

Suitable pH values of the mixture that includes the aqueous base and the first organic stream 165 can be about 9.5 or more, such as from about 9.5 to about 14, such as from about 9.5 to about 13, such as from about 9.5 to 11, from about 10 to about 12, or from about 10 to about 11, though other pH values or ranges are contemplated. Any of the foregoing numbers can be used singly to describe an open-ended range or in combination to describe a close-ended range.

In some embodiments, the aqueous base can include any suitable concentration of alkali metal hydroxide in water. A concentration of the alkali metal hydroxide in the water can be about 1 wt % or more, such as from about 1 wt % to about 20 wt %, such as from about 2 wt % to about 15 wt %, such as from about 3 wt % to about 10 wt %, such as from about 5 wt % to about 9 wt %, such as about 3 wt %, 4 wt %, 5 wt %, 6 wt %, 7 wt %, or 8 wt %, though other values are contemplated. Any of the foregoing numbers can be used singly to describe an open-ended range or in combination to describe a close-ended range.

In some embodiments, a weight ratio of the first organic stream 165 to the aqueous base used for operation 104 can be from about 1:50 to about 50:1, such as from about 1:35 to about 35:1, such as from about 1:20 to about 20:1, such as from about 1:15 to about 15:1, such as from about 1:10 to about 10:1, such as from about 5:1 to about 1:5, such as from about 3:1 to about 1:3, such as from about 2:1 to about 1:2, such as from about 1.2:1 to about 1:1.2, such as about 1:1, though other amounts are contemplated. Any of the foregoing numbers can be used singly to describe an open-ended range or in combination to describe a close-ended range.

During operation 104, the first organic stream 165 and the aqueous base can be charged to a vessel. The resulting mixture can be mixed, stirred, or otherwise agitated under mixing conditions effective to form the second aqueous stream 170 and the second organic stream 175. Mixing conditions of operation 104 can include a temperature of about 15° C. to about 30° C. and an ambient pressure (about 1 atm). Elevated temperatures can be used if desired. However, if elevated temperatures are used during mixing, the mixing temperature should be below the temperature at which the water and organic solvent in the mixture boils.

Mixing conditions of operation 104 can include stirring, mixing, agitating, or combinations thereof by using suitable devices. Suitable devices can include a mechanical stirrer such as an overhead stirrer, a magnetic stirrer (for example, placing a magnetic stir bar in the vessel above a magnetic stirrer), or other suitable devices. For example, a stirrer having a blade or propeller can be rotated by receiving rotational power from a stirring motor to stir the components at suitable rotation speeds, such as from about 50 rpm to about 1,500 rpm, such as from about 75 rpm to about 1,000 rpm, such as from about 100 rpm to about 900 rpm, such as from about 200 rpm to about 800 rpm, such as from about 300 rpm to about 700 rpm, such as from about 400 rpm to about 600 rpm, such as from about 450 rpm to about 550 rpm, such as about 500 rpm. Any of the foregoing numbers can be used singly to describe an open-ended range or in combination to describe a close-ended range. Other rotation speeds are contemplated and can be selected based on the ability to mix the components sufficiently. Mixing conditions of operation 104 can include utilizing a non-reactive gas, such as N₂, Ar, or combinations thereof. For example, a non-reactive gas can be introduced to the first organic stream 165 and aqueous base to degas various components or otherwise remove unwanted gases such as oxygen from the mixture.

Mixing, stirring, or agitating during operation 104 can be performed for any suitable period, such as from about 1 min to about 48 h, such as from about 5 min to about 24 h, such as from about 30 min to about 10 h, such as from about 1 h to about 5 h, such as from about 2 h to about 3 h, though other periods are contemplated. Any of the foregoing numbers can be used singly to describe an open-ended range or in combination to describe a close-ended range.

After mixing, the mixture is separated by suitable liquid-liquid separation techniques, such as decanting, distillation, extraction, extractive distillation, or combinations thereof, among other techniques. After separation, the second aqueous stream 170 and the second organic stream 175 are obtained.

The second aqueous stream 170 can include phenol. The second aqueous stream 170 can optionally include phenolic compounds, other organic compounds, or combinations thereof.

The second organic stream 175 can include phenolic compounds and optionally other organic compounds. The second organic stream 175 can also include substrates used to synthesize epoxy resins. For example, such substrates can include a glycidation substrate different from bisphenol. This substrate can be used to replace bisphenol and to form epoxy resins. In some embodiments, the glycidation substrate comprises a bisphenol. Additionally, or alternatively, the second organic stream 175 can include a curing agent, an accelerator, or combinations thereof. Optionally, the second organic stream 175 can include an advancement resin based on bisphenol A diglycidyl ether, an advancement resin based on bisphenol F diglycidyl ether, or combinations thereof. The curing agents, accelerators, glycidation substrates, advancement resins, or combinations thereof can be used to form an epoxy resin. As such, the second organic stream 175 can include high-value organic compounds. Illustrative, but non-limiting, examples of curing agents present in the second organic stream 175 can include amines, anhydrides, phenolic resins, or combinations thereof, among others. Illustrative, but non-limiting, examples of accelerators present in the second organic stream 175 can include acids, Lewis acids, tertiary amines, phenolic derivatives, or combinations thereof, among others. Illustrative, but non-limiting, examples of glycidation substrates present in the second organic stream 175 can include phenolic-type resins, bisphenolics, amine derivatives, or combinations thereof, among others. In at least one embodiment, the second organic stream further comprises a glycidation substrate, a curing agent, an accelerator, or combinations thereof.

The second organic stream 175 can optionally include phenol. In some embodiments, the second aqueous stream 170 comprises an amount or concentration of phenol that is greater than the amount or concentration of phenol in the second organic stream 175.

In some embodiments, the amount of phenol in the second organic stream 175 is lower than an amount of phenol in the second aqueous stream 170. The phenol in the aqueous stream can exist in its ionic form. In some embodiments, an amount of phenol in the second organic stream 175 can be about 5 wt % or less, 4 wt % or less, 3 wt % or less, 2.5 wt % or less, 2 wt % or less, 1.5 wt % or less, 1 wt % or less, or 0.5 wt % or less, based on a total a total weight of organic compounds present in the second organic stream 175. Any of the foregoing numbers can be used singly to describe an open-ended range or in combination to describe a close-ended range. Other amounts of phenol in the second organic stream 175 are contemplated.

The total weight of organic compounds present in the second organic stream 175 does not exceed 100 wt %. The total weight of organic compounds in the second organic stream 175 does not include the weight of the first organic solvent. Any of the foregoing numbers can be used singly to describe an open-ended range or in combination to describe a close-ended range. The amount of phenol in the second organic stream 175 can be determined by gas-chromatography, mass spectrometry as described in the Examples section.

In some embodiments, the second aqueous stream 170 has an amount or concentration of phenol that is greater than the amount or concentration of phenol in the second organic stream 175. In some embodiments, an amount of phenol in the second aqueous stream 170 can be about 1 wt % or more, such as about 5 wt % or more, such as about 10 wt % or more, such as 20 wt % or more, such as 30 wt % or more, such as about 50 wt % or more, based on a total the total weight of organic compounds present in the second aqueous stream 170. The total weight of organic compounds in the second aqueous stream 170 does not include the weight of the first organic solvent if present.

The amount of phenol in the second aqueous stream 170 can be determined by gas-chromatography, mass spectrometry (GC-MS). Here, and prior to GC-MS, the second aqueous stream 170 can be extracted with a suitable organic solvent, dried with a suitable drying agent such as sodium sulfate or magnesium sulfate, excess solvent removed, and then diluted with a suitable solvent such as tetrahydrofuran for GC-MS analysis.

If desired, the second organic stream 175 can be subjected to operation 104, by which the second organic stream 175 can be contacted with an aqueous base and the resulting mixture separated. After separation, the aqueous layer can be combined with the second aqueous stream 170 and the organic layer can be combined with the second organic stream 175. Subjecting the second organic stream 175 to operation 104 can be utilized to retrieve any remaining phenol, other organic compounds, or combinations thereof in the second organic stream 175. Operation 104 can be performed any suitable number of times such as 1, 2, 3, 4, or more times. Each of the aqueous layers can be retrieved and combined to form the second aqueous stream 170.

In some embodiments, and prior to proceeding with further operations of process 100, solvent such as organic solvent or water can be removed from the second aqueous stream 170, the second organic stream 175, or both. Solvent removal can be accomplished by any suitable technique such as distillation, vacuum distillation, or combinations thereof.

Process 100 further includes contacting the second aqueous stream 170 with an aqueous acid to form a precipitate 185 and a third aqueous stream 180 comprising the phenol at operation 106. The precipitate 185 can include phenolic oligomers, other higher molecular weight phenol derivatives, or combinations thereof. The third aqueous stream 180 can optionally include phenolic compounds and optionally other organic compounds.

In some embodiments, compounds referred to as phenolic oligomers and higher molecular weight phenol derivatives can include compounds having an average molecular weight that is from about twice as large to about 4 times as large as an average molecular weight of compounds present in the composition 190 enriched in phenol depending on the process used to separate the organic recycle stream from the fibers (for example, pyrolysis solvolysis, thermolysis, catalytic depolymerization, or combinations thereof).

For example, compounds present in the oligomeric fraction (corresponding to the precipitate 185) can have a weight-average molecular weight (M_{W}) that is from about 400 g/mol to about 500 g/mol or from about 200 g/mol to about 300 g/mol, and a number-average molecular weight (Mₙ) that is from about 150 g/mol to about 250 g/mol or from about 200 g/mol to about 300 g/mol, though other values are contemplated. In contrast, compounds present in the phenolic fraction (corresponding to the composition 190 enriched in phenol), can have a M_{w} that is from about 100 g/mol to about 200 g/mol, and a Mₙ that is from about 75 g/mol to about 150 g/mol or from about 100 g/mol to about 175 g/mol, though other values are contemplated.

The aqueous acid utilized for operation 106 can comprise, consist essentially of, or consist of an inorganic acid, an organic acid, or combinations thereof.

Any suitable inorganic acid can be utilized. Illustrative, but non-limiting, examples of inorganic acids useful for operation 106 can include hydrochloric acid (HCl), sulfuric acid (H₂SO₄), nitric acid (HNO₃), phosphoric acid (H₃PO₄), boric acid (H₃BO₄), hydrobromic acid (HBr), hydroiodic acid (HI), hydrofluoric acid (HF), perchloric acid (HClO₄), and combinations thereof, among others. In some embodiments, the inorganic acid can comprise HCl, H₂SO₄, HNO₃, H₃PO₄, or combinations thereof. In at least one embodiment, the inorganic acid is selected from the group consisting of HCl, H₂SO₄, HNO₃, H₃PO₄, and combinations thereof.

Any suitable organic acid can be utilized. Organic acids useful for operation 106 can include carboxylic acids (acids containing one or more —CO₂H groups), sulfonic acids (acids containing one or more-SO₃H groups), or combinations thereof. Illustrative, but non-limiting, examples of carboxylic acids can include formic acid (HCO₂H), acetic acid (CH₃CO₂H), propionic acid (CH₃CH₂CO₂H), butyric acid (CH₃CH₂CH₂CO₂H), lactic acid (CH₃CH (OH) CO₂H), sorbic acid (CH₃ (CH)₄CO₂H), fumaric acid ((CO₂H) CH═CHCO₂H), malic acid ((CO₂H) CH₂CH (OH) CO₂H), tartaric acid ((CO₂H) CH (OH) CH (OH) CO₂H), citric acid ((CO₂H) CH₂C (OH) (CO₂H) CH₂CO₂H), benzoic acid (C₆H₅CO₂H), trifluoroacetic acid (CF₃CO₂H), trichloroacetic acid (CCl₃CO₂H), dichloroacetic acid (CHCl₂CO₂H), fluoroacetic acid (FCH₂CO₂H), chloroacetic acid (ClCH₂CO₂H), or combinations thereof. Illustrative, but non-limiting, examples of sulfonic acids can include methanesulfonic acid (CH₃SO₃H), p-toluenesulfonic acid (CH₃C₆H₄SO₃H), trifluoromethanesulfonic acid (CF₃SO₃H), benzenesulfonic acid (C₆H₅SO₃H), or combinations thereof. Other organic acids can include picric acid ((O₂N)₃C₆H₂OH). Other organic acids are contemplated.

In at least one embodiment, the organic acid is selected from the group consisting of formic acid, acetic acid, citric acid, oxalic acid, and combinations thereof.

The aqueous acid can be added to the second aqueous stream 170 until the pH reaches a desired value and maintains that value such that phenolates present in the second aqueous stream can convert to their corresponding phenols and phenolic compounds. Here, and in some embodiments, operation 106 can include adding the aqueous acid to the second aqueous stream 170 to form a mixture having a desired pH. At operation 106, higher molecular weight phenol derivatives and oligomers can precipitate from the mixture.

Suitable pH values of the mixture that includes the aqueous acid and the second aqueous stream 170 can be about 7.5 or less, such as from about 1 to about 7, such as from about 1 to about 6.5, such as from about 1 to about 5, about 2 to about 4, about 1 to about 3, or from about 2 to about 3, though other pH values or ranges are contemplated. Any of the foregoing numbers can be used singly to describe an open-ended range or in combination to describe a close-ended range. In at least one embodiment, the pH value of the mixture that includes the aqueous acid and the second aqueous stream is about 5 or less.

In some embodiments, the aqueous acid used for operation 106 can have any suitable concentration in water. A concentration of the acid in the water can be about 1 wt % or more, such as from about 1 wt % to about 20 wt %, such as from about 2 wt % to about 15 wt %, such as from about 3 wt % to about 10 wt %, such as from about 5 wt % to about 9 wt %, such as about 3 wt %, 4 wt %, 5 wt %, 6 wt %, 7 wt %, or 8 wt %, though other values are contemplated. Any of the foregoing numbers can be used singly to describe an open-ended range or in combination to describe a close-ended range.

In some embodiments, a weight ratio of the second aqueous stream 170 to aqueous acid used for operation 106 can be from about 1:50 to about 50:1, such as from about 1:35 to about 35:1, such as from about 1:20 to about 20:1, such as from about 1:15 to about 15:1, such as from about 1:10 to about 10:1, such as from about 5:1 to about 1:5, such as from about 3:1 to about 1:3, such as from about 2:1 to about 1:2, such as from about 1.2:1 to about 1:1.2, such as about 1:1, though other amounts are contemplated. Any of the foregoing numbers can be used singly to describe an open-ended range or in combination to describe a close-ended range.

During operation 106, the second aqueous stream 170 and the aqueous acid can be charged to a vessel. The resulting mixture can be mixed, stirred, or otherwise agitated under mixing conditions effective to form the precipitate 185 comprising phenolic oligomers and a third aqueous stream comprising phenol. Mixing conditions of operation 106 can include a temperature of about 15° C. to about 30° C. and an ambient pressure (about 1 atm). Elevated temperatures can be used if desired. However, if elevated temperatures are used during mixing, the mixing temperature should be below the temperature at which the water and organic solvent in the mixture boils.

Mixing conditions of operation 106 can include stirring, mixing, agitating, or combinations thereof by using suitable devices. Suitable devices can include a mechanical stirrer such as an overhead stirrer, a magnetic stirrer (for example, placing a magnetic stir bar in the vessel above a magnetic stirrer), or other suitable devices. For example, a stirrer having a blade or propeller can be rotated by receiving rotational power from a stirring motor to stir the components at suitable rotation speeds, such as from about 50 rpm to about 1,500 rpm, such as from about 75 rpm to about 1,000 rpm, such as from about 100 rpm to about 900 rpm, such as from about 200 rpm to about 800 rpm, such as from about 300 rpm to about 700 rpm, such as from about 400 rpm to about 600 rpm, such as from about 450 rpm to about 550 rpm, such as about 500 rpm. Any of the foregoing numbers can be used singly to describe an open-ended range or in combination to describe a close-ended range. Other rotation speeds are contemplated and can be selected based on the ability to mix the components sufficiently. Mixing conditions of operation 106 can include utilizing a non-reactive gas, such as N₂, Ar, or combinations thereof. For example, a non-reactive gas can be introduced to the second aqueous stream 170, and the aqueous acid to degas various components or otherwise remove unwanted gases (for example, oxygen) from the mixture.

Mixing, stirring, or agitating during operation 106 can be performed for any suitable period, such as from about 1 min to about 48 h, such as from about 5 min to about 24 h, such as from about 30 min to about 10 h, such as from about 1 h to about 5 h, such as from about 2 h to about 3 h, though other periods are contemplated. Any of the foregoing numbers can be used singly to describe an open-ended range or in combination to describe a close-ended range.

After mixing, the mixture can be subjected to any suitable separation technique such as solid/liquid techniques including mechanical or gravity separation, such as filtration, vacuum filtration, centrifuges, decanters, decanter centrifuges, combinations thereof, among other techniques. Separation can be aided by pressing of the solid filter cake that forms. The separation can be performed one or more times. Filtration can be accomplished using a porous surface to draw the filtrate (liquid) from the mixture to one side of the porous surface, and leaving the substrate source as retentate (solid, precipitate, or filter cake) on the opposite side of the porous surface. As an example, the mixture formed at operation 106 can be separated via, for example, filtration to provide a filtrate comprising the third aqueous stream 180, and the retentate (for example, precipitate 185).

The porous surface can be a membrane or frit made of any suitable material such as ceramic, glass, or other materials. The pores of the porous membrane can be selected to separate substrates of specific sizes or ranges (for example, weight-average molecular weights or ranges) from the substrate source. The separation process of operation 106 can be performed one or more times. After the desired number of separations, the precipitate 185 can be used for conversion processes such as a monomer or as a co-monomer with phenol in epoxy resin synthesis. The filtrate comprising the third aqueous stream 180 can be submitted to operation 108, as further described below.

If desired, the precipitate 185 can be subjected to operation 106, by which the precipitate 185 can be contacted with an aqueous acid and the resulting mixture separated. After separation, the aqueous layer can be combined with the third aqueous stream 180. Subjecting the precipitate 185 to operation 106 can be utilized to retrieve any remaining phenol, other organic compounds, or combinations thereof, in the precipitate 185. Operation 106 can be performed any suitable number of times such as 1, 2, 3, 4, or more times. Each of the aqueous layers can be retrieved and combined to form the third aqueous stream 180.

In some embodiments, and prior to proceeding with further operations of process 100, solvent such as organic solvent or water can be removed from the precipitate 185, the third aqueous stream 180, or both. Solvent removal can be accomplished by suitable techniques such as distillation, vacuum distillation, or combinations thereof.

Process 100 further includes contacting the third aqueous stream 180 with a second organic solvent to form a composition 190 at operation 108. The composition 190 can be enriched in phenol. The composition 190 has a second concentration of phenol, where the second concentration of phenol can be greater than the first concentration of phenol. That is, the concentration of phenol in the composition 190 formed by process 100 can be greater than the concentration of phenol in the feedstock 155. Operation 108 also produces a fourth aqueous stream 195.

The composition 190 enriched in phenol comprises, consists essentially of, or consists of phenol, and optionally, one or more additional components. The optional one or more additional compounds can include phenolic compounds represented by formula (II). In some examples, the optional one or more additional components can include p-cumenol, 4,4′-(1-methylethylidene) bis-phenol, 4-isopropyl-3-methylphenol, second organic solvent, or combinations thereof.

The second organic solvent utilized for operation 108 can include those organic solvents described above. In some embodiments, the second organic solvent utilized for operation 108 can comprise, consist essentially of, or consist of halogenated solvent, ether solvent, ketone solvent, ester solvent, or combinations thereof. In at least one embodiment, the second organic solvent utilized for operation 108 is selected from the group consisting of halogenated solvent, ether solvent, ketone solvent, ester solvent, and combinations thereof. In some embodiments, the second organic solvent comprises an aprotic solvent.

In some embodiments, the second organic solvent utilized for operation 108 can comprise, consist essentially of, or consist of dichloromethane, chloroform, methyl tert-butyl ether, ethyl acetate, toluene, benzene, hexane, cyclohexane, and combinations thereof. In at least one embodiment, the second organic solvent utilized for operation 108 is selected from the group consisting of dichloromethane, chloroform, methyl tert-butyl ether, ethyl acetate, toluene, benzene, hexane, cyclohexane, or combinations thereof.

In at least one embodiment, each of the first and second organic solvents, independently, comprise an aprotic solvent. In some embodiments, each of the first and second organic solvents are independently selected from the group consisting of halogenated solvent, ether solvent, ketone solvent, ester solvent, and combinations thereof. In at least one embodiment, each of the first and second organic solvents are independently selected from the group consisting of dichloromethane, chloroform, methyl tert-butyl ether, ethyl acetate, toluene, benzene, hexane, cyclohexane, and combinations thereof. In some embodiments, each of the first and second aprotic solvents are independently selected from the group consisting of dichloromethane, chloroform, methyl tert-butyl ether, ethyl acetate, and combinations thereof.

A weight ratio of the third aqueous stream 180 to second organic solvent used for operation 108 can be from about 1:50 to about 50:1, such as from about 1:35 to about 35:1, such as from about 1:20 to about 20:1, such as from about 1:15 to about 15:1, such as from about 1:10 to about 10:1, such as from about 5:1 to about 1:5, such as from about 3:1 to about 1:3, such as from about 2:1 to about 1:2, such as from about 1.2:1 to about 1:1.2, such as about 1:1, though other amounts are contemplated. Any of the foregoing numbers can be used singly to describe an open-ended range or in combination to describe a close-ended range.

During operation 108, the third aqueous stream 180 and second organic solvent can be charged to a vessel. The resulting mixture can be mixed, stirred, or otherwise agitated under mixing conditions effective to form the fourth aqueous stream 195 and the composition 190 enriched in phenol. Mixing conditions of operation 108 can include a temperature of about 15° C. to about 30° C. and an ambient pressure (about 1 atm). Elevated temperatures can be used if desired. However, if elevated temperatures are used during mixing, the mixing temperature should be below the temperature at which the water and the second organic solvent boils.

Mixing conditions of operation 108 can include stirring, mixing, agitating, or combinations thereof by using suitable devices. Suitable devices can include a mechanical stirrer such as an overhead stirrer, a magnetic stirrer (for example, placing a magnetic stir bar in the vessel above a magnetic stirrer), or other suitable devices. For example, a stirrer having a blade or propeller can be rotated by receiving rotational power from a stirring motor to stir the components at suitable rotation speeds, such as from about 50 rpm to about 1,500 rpm, such as from about 75 rpm to about 1,000 rpm, such as from about 100 rpm to about 900 rpm, such as from about 200 rpm to about 800 rpm, such as from about 300 rpm to about 700 rpm, such as from about 400 rpm to about 600 rpm, such as from about 450 rpm to about 550 rpm, such as about 500 rpm. Any of the foregoing numbers can be used singly to describe an open-ended range or in combination to describe a close-ended range. Other rotation speeds are contemplated and can be selected based on the ability to mix the components sufficiently. Mixing conditions of operation 108 can include utilizing a non-reactive gas, such as N₂, Ar, or combinations thereof. For example, a non-reactive gas can be introduced to the third aqueous stream 180 and second organic solvent to degas various components or otherwise remove unwanted gases (for example, oxygen) from the mixture.

Mixing, stirring, or agitating during operation 108 can be performed for any suitable period, such as from about 1 min to about 48 h, such as from about 5 min to about 24 h, such as from about 30 min to about 10 h, such as from about 1 h to about 5 h, such as from about 2 h to about 3 h, though other periods are contemplated. Any of the foregoing numbers can be used singly to describe an open-ended range or in combination to describe a close-ended range.

After mixing, the mixture of the fourth aqueous stream 195 and the second organic solvent is separated by suitable liquid-liquid separation techniques, such as decanting, distillation, extraction, extractive distillation, or combinations thereof, among other techniques. After separation, the fourth aqueous stream 195 and the composition 190 enriched in phenol is obtained. In some embodiments, the fourth aqueous stream 195 can optionally include phenolic compounds, other organic compounds, or combinations thereof.

If desired, the fourth aqueous stream 195 can be subjected to operation 108, by which the fourth aqueous stream 195 can be contacted with a second organic solvent and the resulting mixture separated. After separation, the organic layer can be combined with the composition 190 enriched in phenol. Subjecting the fourth aqueous stream 195 to operation 108 can be utilized to retrieve any remaining phenol, other organic compounds, or combinations thereof, in the fourth aqueous stream 195. Operation 108 can be performed any suitable number of times such as 1, 2, 3, 4, or more times. Each of the organic layers can be retrieved and combined to form the composition 190 enriched in phenol.

If desired, solvent such as organic solvent or water can be removed from the composition 190 enriched in phenol, the fourth aqueous stream 195, or both. Solvent removal can be accomplished by suitable techniques such as distillation, vacuum distillation, or combinations thereof. The phenol present in the composition 190 enriched in phenol can be referred to as “recycled phenol”.

Referring back to FIG. 1B, the dashed box 172 indicates that the precipitate 185 comprising phenolic oligomers and the composition 190 enriched in phenol can be used as a monomer, a co-monomer, or both for various conversion processes such as epoxy resin synthesis and novolak synthesis. When the precipitate 185 comprising phenolic oligomers, the composition 190 enriched in phenol, or combinations thereof are used as a co-monomer, the other monomer can include phenol (such as a non-recycled phenol, for example, commercial grade phenol).

In addition, and as described above, the second organic stream 175 can include phenolic compounds and optionally other organic compounds. The second organic stream 175 can also include substrates used to synthesize epoxy resins (such as a glycidation substrate different from bisphenol). Such substrates can be used to replace bisphenol and be used to form epoxy resins. Additionally, or alternatively, second organic stream 175 can include a curing agent, an accelerator, or combinations thereof. The curing agents, accelerators, and glycidation substrates can be used to form an epoxy resin. As such, the second organic stream 175 can include high-value organic compounds.

Accordingly, embodiments of the process 100 can enable recycling of phenol, phenolic compounds, curing agents, accelerators, and glycidation substrates from feedstocks comprising solvolysis oil, pyrolysis oil, or combinations thereof. The phenol, phenolic compounds, curing agents, accelerators, and glycidation substrates separated from solvolysis oil and pyrolysis oil can be used for conversion processes such as conversion to novolak resins among other applications.

As described above, conventional technologies have been unable to address the re-use or recyclability of thermosets present in fiber-reinforced thermoset composites (such as epoxy resin composites and other resin composites). The conventional wisdom has been to view such thermosets as non-recyclable. In contrast, processes described herein can enable sustainable recycling technologies for fiber-reinforced thermoset composites such as epoxy resin composites and other resin composites. Here, processes of the present disclosure can enable retrieval of valuable products such as phenol, phenolic compounds, and other organic compounds from solvolyzed fiber-reinforced thermoset composites (solvolysis oil), pyrolyzed fiber-reinforced thermoset composites (pyrolysis oil), thermolyzed fiber-reinforced thermoset composites (thermolysis oil), catalytically decomposed fiber-reinforced thermoset composites, among other recycle streams. As a consequence, processes described herein can enable sustainable recycling technologies for fiber-reinforced thermoset composites such as epoxy resin composites and other resin composites. In addition, processes described herein can also mitigate landfilling issues related to fiber-reinforced thermoset composites.

### Conversion Process

Embodiments of the present disclosure also relate to processes for producing a phenol-formaldehyde resin (for example, a novolak resin) from the recycled phenol, phenolic compounds, or combinations thereof. Again, the recycled phenol refers to the phenol obtained from process 100 or flow diagram 150, for example, the phenol present in the composition 190 enriched in phenol. All phenolic derivatives present in the composition 190 enriched in phenol can react with formaldehyde.

The recycled phenol, phenolic compounds, or combinations thereof can be utilized as a monomer or a co-monomer to form novolak resins. Additionally, or alternatively, the precipitate 185 comprising phenolic oligomers can be utilized as a monomer or as a co-monomer with phenol to form novolak resins. Novolak resins are polymers derived from a phenol and formaldehyde. Besides phenol, cresols such as o-cresol, m-cresol, p-cresol, or combinations thereof, and other phenolic compounds present in the composition enriched in phenol can be converted to novolak resins.

The recycled phenol, phenolic compounds, or combinations thereof can be converted to a novolak resin by any suitable process. Schemes 1 and 2 show illustrative, but non-limiting, general reaction scheme for forming a novolak resin. In Scheme 1, compound (I) is phenol, compound (V) is a source of formaldehyde, and formula (VI-A) is a novolak resin product. In Scheme 2, the compound of formula (II) represents phenolic compounds, compound (V) is a source of formaldehyde, and formula (VI-B) is a novolak resin product. As described above, the phenolic compounds of formula (II) can be present in the composition 190 enriched in phenol.

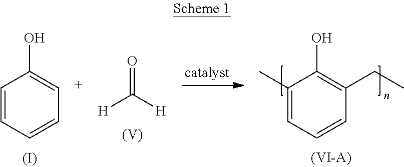

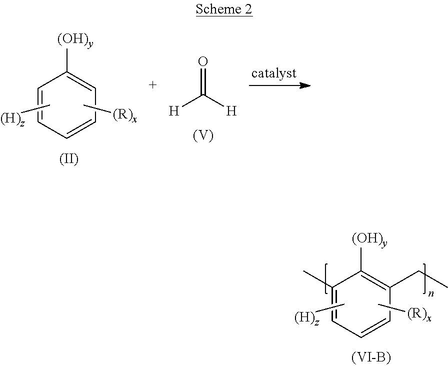

The conversion process generally includes reacting a mixture of a formaldehyde or a source of formaldehyde, a catalyst, and a phenol, under reaction conditions, to form a reaction product of formula (VI-A). Besides phenol (compound of formula (I)), phenolic compounds of formula (II) can be utilized to make the corresponding novolak resin as shown in Scheme 2. Such phenolic compounds can be present in the composition 190 enriched in phenol. Although embodiments of the conversion process are described with respect to formaldehyde, it is contemplated that other aldehydes, as well as ketones, can be used, as further described below with respect to aldehydes and ketones of formula (VII). In addition, although embodiments of the conversion process are described with respect to using the composition 190 enriched in phenol, it is contemplated that the precipitate 185 comprising phenolic oligomers can be utilized as a monomer or as a co-monomer with phenol to form novolak resins.

In some embodiments, the composition 190 enriched in phenol (the composition containing recycled phenol, phenolic compounds of formula (II), or combinations thereof) can be subjected to conversion conditions effective to form a novolak resin. In these and other embodiments, a non-recycled phenol such as commercial grade phenol can optionally be added. In conversion processes that include both the composition 190 enriched in phenol and non-recycled phenol, a weight ratio of composition 190 to the non-recycled phenol can be any suitable weight ratio, such as from about 50:1 to about 1:50, such as from about 35:1 to about 1:35, such as from about 20:1 to about 1:20, such as from about 15:1 to about 1:15, such as from about 10:1 to about 1:10, such as from about 5:1 to about 1:5, such as from about 3:1 to about 1:3, such as from about 2:1 to about 1:2, though other amounts are contemplated. Any of the foregoing numbers can be used singly to describe an open-ended range or in combination to describe a close-ended range. In at least one embodiment, a weight ratio of composition 190 to the non-recycled phenol can be from about 90:10 to about 10:90, such as from about 8:2 to about 2:8, such as from about 7:3 to about 3:7, such as from about 6:4 to about 4:6, such as about 1:1, though other amounts are contemplated. Any of the foregoing numbers can be used singly to describe an open-ended range or in combination to describe a close-ended range.

Besides formaldehyde, paraformaldehyde ((CH₂O)ₙ) can be used as a source of formaldehyde. Additionally, or alternatively, formalin (an aqueous solution of formaldehyde) can be utilized as a source of formaldehyde. In some embodiments, the formaldehyde (or source of formaldehyde) is introduced to a mixture comprising a phenol (compound of formula (I)), a phenolic compound of formula (II), or combinations thereof. The mixture can also include solvent, catalyst, or both, among other components.

Other aldehydes, as well as ketones, are also contemplated, such as those represented by formula (VII):

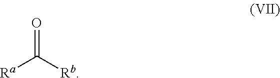

In formula (VII), each of R^{a} and R^{b} can be, independently, hydrogen, an unsubstituted hydrocarbyl, a substituted hydrocarbyl, or a functional group comprising at least one element from Group 13-17 of the periodic table of the elements. Suitable R groups for each of R^{a} and R^{b} formula (VII) can include those described above with respect to formula (II). Illustrative, but non-limiting, examples of unsubstituted hydrocarbyl include an alkyl group having from 1 to 20 carbon atoms such as methyl, ethyl, n-propyl, isopropyl, n-butyl, iso-butyl, sec-butyl, and tert-butyl, pentyl, hexyl, heptyl, octyl, ethyl-2-hexyl, isooctyl, nonyl, n-decyl, isodecyl, or isomers thereof; a cycloaliphatic group having from 3 to 20 carbon atoms such as, for example, cyclopentyl or cyclohexyl; an aromatic group having from 6 to 20 carbon atoms such as, for example, phenyl or naphthyl; or any combination thereof.

Each of R^{a} and R^{b} of formula (VII) can have, independently, any suitable number of carbon atoms such as from 1 to 20 carbon atoms, such as from 1 to 10 carbon atoms, such as from 1 to 5 carbon atoms, such as from 1 to 4 carbon atoms. In some embodiments, the number of carbon atoms in each of R^{a} and R^{b} of formula (VII) can be, independently, 1, 2, 3, 4, 5, 6, 7, 8, 9, 10, 11, 12, 13, 14, 15, 16, 17, 18, 19, or 20. Each of the foregoing numbers can be preceded by the word “about,” “at least about,” “less than about,” or “more than about,” and any of the foregoing numbers can be used singly to describe an open-ended range or in combination to describe a close-ended range. Each of R^{a} and R^{b} of formula (VII) group can be, independently, linear or branched, saturated or unsaturated, cyclic or acyclic, aromatic or not aromatic. Regarding saturation, each of R^{a} and R^{b} of formula (VII) can be, independently, fully saturated, partially unsaturated, or fully unsaturated.

Illustrative, but non-limiting, examples of aldehydes include formaldehyde, acetaldehyde, crotonaldehyde, pentanal, butanal, 3-methyl-butenal, acrolein, benzaldehyde, furfural, glyoxal, derivatives thereof, and combinations thereof.

Illustrative, but non-limiting, examples of ketones include propanone (acetone), acetophenone (methyl phenyl ketone), benzophenone (diphenyl ketone), 2-pentanone (methyl propyl ketone), 3-methyl-2-butanone (methyl isopropyl ketone), 3-hexanone (ethyl propyl ketone), derivatives thereof, or combinations thereof.

A total amount of the composition 190 enriched in phenol and the non-recycled phenol (if used) to the amount of formaldehyde used for the conversion reaction is referred to as a phenol/formaldehyde (P/F) weight ratio. The P/F ratio for the conversion process can be from about 1:0.3 to about 1:0.8, such as from about 1:0.4 to about 1:0.6, such as about 1:0.5, though other ratios are contemplated. Any of the foregoing numbers can be used singly to describe an open-ended range or in combination to describe a close-ended range.

The catalyst for the conversion process can include any suitable acid catalyst or base catalyst such as Brønsted acids and Brønsted bases. Illustrative, but non-limiting, examples of the catalyst include organic acids, inorganic acids, or combinations thereof. Organic acids include, but are not limited to, methanesulfonic acid, p-toluenesulfonic acid, trifluoroacetic acid, oxalic acid (CAS No. 144-62-7), or combinations thereof, among others. Illustrative, but non-limiting, examples of inorganic acids include hydrochloric acid, sulfuric acid, phosphoric acid, or combinations thereof, among others. Acid catalysts can be removed by heating the reaction product comprising the novolak resin of formula (VI-A), the novolak resin of formula (VI-B), or combinations thereof at high temperatures. For example, oxalic acid decomposes to carbon dioxide at temperatures of about 160° C. or more, for example, about 165° C. or more. Additionally, or alternatively, acid catalysts can also be removed from the reaction product comprising the novolak resin of formula (VI-A), the novolak resin of formula (VI-B), or combinations thereof by distillation of the reaction product, by neutralization with sodium hydroxide (NaOH), or combinations thereof.

Illustrative, but non-limiting, examples of base catalysts include sodium hydroxide, potassium hydroxide, sodium carbonate, sodium hydrogen carbonate, sodium bicarbonate, potassium carbonate, potassium bicarbonate, basic amines, or combinations thereof. The base catalyst used can be an aqueous solution of about 5% to about 50% by weight, such as about 10% to about 40% by weight, such as about 20% to about 30% by weight. Any of the foregoing numbers can be used singly to describe an open-ended range or in combination to describe a close-ended range.

The mixture comprising the phenol (compound of formula (I)), a phenolic compound of formula (II), or combinations thereof can also include one or more solvents. Suitable solvents include an organic solvent. Organic solvents can include, but are not limited to aliphatic hydrocarbons, cycloaliphatic hydrocarbons, aromatic hydrocarbons, alcohols, ethers, esters, nitriles, or combinations thereof. Illustrative, but non-limiting, examples of organic solvents include tert-butylbenzene (tBB), toluene, ethylbenzene, xylene (one or more of 1,2-dimethylbenzene, 1,3-dimethylbenzene, or 1,4-dimethylbenzene), 1,3,5-trimethylbenzene (also known as mesitylene), decane, the monomethyl ether of diethylene glycol, ethylene glycol of monobutyl ether, tetrahydrofuryl alcohol, ethylene glycol monomethyl ether, ethyl acetate, isopropyl acetate, butyl acetate, amyl acetate, isomers thereof, or combinations thereof. Other solvents are contemplated. In some embodiments, the organic solvent includes tert-butylbenzene (tBB), toluene, ethylbenzene, a xylene, 1,3,5-trimethylbenzene, isomers thereof, or mixtures thereof.

In some embodiments, the solvent can be selected based on a boiling point that can be utilized to remove or neutralize the catalyst used. For example, the solvent can be selected to have a boiling point (at 100 kPa (absolute)) of about 100° C. or more in order to decompose or neutralize an acid or base. As an illustrative, but non-limiting, example, solvents boiling between about 160° C. and about 170° C. (such as tert-butylbenzene, 1,3,5-trimethylbenzene, or other solvents) can be utilized with oxalic acid catalyst, and after formation of the bisphenol, the oxalic acid can be removed from the bisphenol/solvent/oxalic acid mixture at temperatures of about 165° C. to about 170° C. Heating at this temperature results in decomposition and sublimation of oxalic acid.

In some examples, the solvent can have a boiling point (at 100 kPa (absolute)) that is from about 100° C. to about 210° C., such as from about 110° C. to about 200° C., such as from about 120° C. to about 190° C., such as from about 130° C. to about 180° C., such as from about 140° C. to about 170° C., such as from about 150° C. to about 160° C. or from about 165° C. to about 170° C. Any of the foregoing numbers can be used singly to describe an open-ended range or in combination to describe a close-ended range.

The novolak resin of formula (VI-A), the novolak resin of formula (VI-B), or combinations thereof can be made by feeding the composition 190 enriched in phenol, source of formaldehyde, catalyst, and solvent to a reactor. As described above, non-recycled phenol can additionally be utilized. The resultant mixture can be reacted under reaction conditions effective to form the novolak resin of formula (VI-A), the novolak resin of formula (VI-B), or combinations thereof.

Reaction conditions can include a reactor temperature that is from about 50° C. to about 200° C., such as from about 70° C. to about 120° C., such as from about 90° C. to about 100° C., though other temperatures are contemplated. Any of the foregoing numbers can be used singly to describe an open-ended range or in combination to describe a close-ended range. The reactor temperature can be the temperature (or temperature range) at which the reaction mixture boils, an azeotropic boiling point (or range) of water-solvent combination. In some examples, using tBB as solvent under atmospheric conditions, the reactor temperature can be from about 75° C. to about 100° C., such as from about 80° C. to about 96° C., such as about 96° C. Reactor temperature is the temperature monitored by a temperature probe.

Reaction conditions can also include a reactor pressure as measured in units of absolute pressure. The reactor pressure can be from about 100 kPa (absolute) to about 450 kPa (absolute), such as from about 105 kPa (absolute) to about 180 kPa (absolute), such as from about 110 kPa (absolute) to about 120 kPa (absolute), though other pressures are contemplated. Any of the foregoing numbers can be used singly to describe an open-ended range or in combination to describe a close-ended range. In at least one embodiment the reactor pressure of reaction conditions can be from about 100 kPa (absolute) to about 180 kPa (absolute), such as from about 100 kPa (absolute) to about 120 kPa (absolute). Any of the foregoing numbers can be used singly to describe an open-ended range or in combination to describe a close-ended range. The pressure (or pressure ranges) can be selected to match the temperature (or temperature ranges) in terms of the reaction mixture boiling for a pressure ≥100 kPa (absolute) and a temperature ≥96° C.

Reaction conditions also include a reaction period. The period for reaction can be any suitable amount of time, such as from about 0.5 to 20 hours, such as from about 1 to about 10 hours, such as from about 2 to about 5 hours, though other periods are contemplated. Any of the foregoing numbers can be used singly to describe an open-ended range or in combination to describe a close-ended range.

After the reaction is deemed complete, water and formaldehyde can be removed from the reaction product comprising the novolak resin of formula (VI-A), the novolak resin of formula (VI-B), or combinations thereof. Removal of water and formaldehyde can be performed by, for example, distillation (under suitable conditions) of the reaction product.

In some embodiments, the temperature at which the removal of water and formaldehyde occurs is performed at a temperature lower than the boiling point of the solvent. In cases where solvent evaporates or distills off while removing the formaldehyde and water, the solvent can be separated from the water phase in, for example, a phase separator of a condenser and phase separation vessel, and then returned to the reactor that includes the reaction product.

After the conversion reaction, a reaction product comprising the novolak resin of formula (VI-A), the novolak resin of formula (VI-B), or combinations thereof is obtained.

In some embodiments, the conversion reaction for converting the composition 190 enriched in phenol to the phenol-formaldehyde resin comprises forming a mixture comprising the composition enriched in the phenol, an aldehyde or ketone, and a catalyst; and reacting the mixture to form a phenol-formaldehyde resin.

In some embodiments, the precipitate 185 that includes phenolic oligomers can be utilized as a monomer or a co-monomer (with phenol) to form novolak resins. Additionally, or alternatively, the precipitate 185 that includes phenolic oligomers can be reacted with an epoxy resin in an advancement reaction. In at least one embodiment, the second organic stream 175 (which can include a curing agent, an accelerator, or combinations thereof) can be added to an epoxy resin. Overall, various fractions obtained from embodiments described herein include valuable products that can be utilized for a variety of reactions.

The following examples are put forth so as to provide those of ordinary skill in the art with a complete disclosure and description of how to make and use embodiments of the present disclosure, and are not intended to limit the scope of embodiments of the present disclosure. Efforts have been made to ensure accuracy with respect to numbers used but some experimental errors and deviations should be accounted for.

### EXAMPLES

### Test Methods

### Characterization

Infrared spectroscopy was performed to determine functional groups present in various streams of processes described herein. Infrared spectra were obtained using a FTIR spectrometer (Bruker Alpha) equipped with a SB-Diamond attenuated total reflectance. 32 scans were run and averaged.

Gel permeation chromatography was performed to determine molecular weight distributions of products within various streams of processes described herein. Gel permeation chromatography chromatograms were obtained using a LaChrom Elite (VWR Hitachi). The set-up included a UV-Vis detector (280 nm), and a 1× PLgel Mixed-E column (300×7.5 internal diameter (I.D.)). Samples for gel permeation chromatography were diluted in tetrahydrofuran (THF). Samples were run at 1 mL/min.

Gas chromatography-mass spectrometry (GC-MS) was performed to determine amount of phenol in the second organic stream. GC-MS was obtained using an Agilent 8890 GC with Agilent 5977B mass selective detector equipped with an HP-5 ms ((5%-phenyl)-methylpolysiloxane phase) capillary column GC column (30 m length, 0.25 mm I.D., 0.25 μm film thickness; part number: 19091S-433UI). Gas chromatography-flame ionization detection (GC-FID) was performed using an Agilent 8890 GC equipped with a flame ionization detector and DB-1 GC column (60 m length, 0.32 mm I.D., 1 μm film thickness; part number: 123-1063).

Ultra-high-performance liquid chromatography (UHPLC) was performed to determine phenol and p-toluene sulfonic acid content. UHPLC was also performed to determine the presence of novolak resin products after subjecting various fractions to novolak resin synthesis. UHPLC was performed using an ultra-high-performance liquid chromatography-photodiode array (UHPLC-PDA) system. The system included a Waters Acquity UHPLC H-Class, equipped with an Acquity UPLC BEH Phenyl column and PDA detector. Samples were run using a THF/Water system.

### Example 1: Example Process to Form a Composition Enriched in Phenol

Separating phenol and other organic fractions from recycle streams by embodiments described herein were performed. Two feedstocks (for example, feedstock 155) were subjected to processes described herein. One feedstock included a pyrolysis oil and the other feedstock included a solvolysis oil.

To a feedstock (about 50 g) was added a first organic solvent (dichloromethane, DCM, about 50 mL). The resultant mixture was then mixed and washed with water (3×100 mL) to remove organic acids, and the aqueous layers were combined. The collected fractions included a first aqueous stream 160 (containing organic acids) and a first organic stream 165.

The first organic stream 165 was then washed and mixed with an aqueous base (5% sodium hydroxide in water; 3×100 mL) to concentrate the base soluble compounds, and the aqueous layers were combined. The collected fractions included a second aqueous stream 170 (containing the base soluble compounds) and a second organic stream 175 the second aqueous stream 170. In this stage, the washing and mixing was performed until aqueous layers became clear.

### Example 1A: Organic Fraction

Prior to further use, the second organic stream 175 was washed with water (3×100 mL), dried over sodium sulfate (Na₂SO₄), filtered, and the solvent removed by vacuum. The “organic fraction” for the Examples corresponds to the second organic stream 175.

### Example 1B: Phenolic Fraction

Hydrochloric acid (HCl, 12 M, 37% in water, 40 mL) was added to the second aqueous stream 170 until a pH of less than about 5 was obtained. A solid residue (for example, precipitate 185) formed. The precipitate 185 was filtered and the third aqueous stream 180 was retrieved.

The third aqueous stream 180 was mixed and extracted with a second organic solvent (DCM; 3×100 mL). The organic layers were combined to form the composition 190 enriched in phenol.

Prior to further use, the composition 190 enriched in phenol was dried over Na₂SO₄, filtered, and the solvent removed by vacuum. The “phenolic fraction” for the Examples corresponds to the composition 190 enriched in phenol.

### Example 1C: Oligomeric Fraction

Prior to further use, the solid residue (for example, precipitate 185) was dissolved in DCM (50 mL) or acetone (50 mL), and washed with water (3×100 mL) or until the aqueous phase became clear. The organic layers were combined, dried over Na₂SO₄, filtered, and the solvent removed by vacuum. The “oligomeric fraction” for the Examples corresponds to the precipitate 185.

Table 1-1 shows generic data for the primary components of the crude solvolysis oil (raw material feedstock) and three separated fractions-organic fraction, oligomeric fraction, and phenolic fraction. The amounts of components, in area percent, of the individual compositions were determined by GC-FID×GC-MS. Phenol had a retention time of about 4.1-4.2 minutes.

TABLE 1-1

| Sample | Component | Area percent |
|---|---|---|
| Solvolysis oil | Phenol | 45 |
| (raw material) | Remainder balance | 55 |
| Organic | Phenol | 2 |
| fraction | Remainder balance including | 98 |
| | phenolic compounds | |
| Oligomeric | Phenol | 15 |
| fraction | Remainder balance including | 85 |
| | phenolic oligomers | |
| Phenolic | Phenol | 85 |
| fraction | Remainder balance including | 15 |
| | phenolic compounds | |

Overall, the data in Table 1-1 indicates that processes described herein can be utilized to separate various valuable fractions (for example, a phenolic fraction, an oligomeric fraction, and an organic fraction) from crude solvolysis oil feedstocks. For example, and as shown in Table 1-1, the raw material included about 45% phenol and 55% of other compounds. The amount of phenol remained low in the organic fraction (about 2%) and the oligomeric fraction (about 17%). The phenolic fraction (which corresponds to the composition 190 enriched in phenol) contained about 85% recycled phenol and other compounds that can be used to form, for example, novolak resins. Further, the oligomeric fraction-which corresponds to the precipitate 185) contains phenolic oligomers as well as phenolic compounds that can be used to form novolak resins or can be reacted with an epoxy resin in an advancement reaction.

In some embodiments, the precipitate 185 that includes phenolic oligomers can be utilized as a monomer or a co-monomer (with phenol) to form novolak resins. Additionally, or alternatively, the precipitate 185 that includes phenolic oligomers can be reacted with an epoxy resin in an advancement reaction. In at least one embodiment, the second organic stream 175 (which can include a curing agent, an accelerator, or combinations thereof) can be added to an epoxy resin. Overall, various fractions obtained from embodiments described herein include valuable products that can be utilized for a variety of reactions and other uses.

Table 1-2 shows selected data for the primary components of the crude pyrolysis oil (raw material feedstock) and three separated fractions-organic fraction, oligomeric fraction, and phenolic fraction. The amounts of components, in area percent, of the individual compositions were determined by GC-FID×GC-MS. Phenol had a retention time of about 4.1-4.2 minutes.

TABLE 1-2

| Sample | Component | Area percent |
|---|---|---|
| Pyrolysis oil | Phenol | 27 |
| (raw material) | Remainder balance | 73 |
| Organic | Phenol | 0 |
| fraction | Remainder balance including | 100 |
| | phenolic compounds | |
| Oligomeric | Phenol | 30 |
| fraction | Remainder balance including | 70 |
| | phenolic oligomers | |
| Phenolic | Phenol | 0 |
| fraction | Remainder balance including | 100 |
| | phenolic compounds | |

Overall, the data in Table 1-2 indicates that processes described herein can be utilized to separate various valuable fractions (for example, a phenolic fraction, an oligomeric fraction, and an organic fraction) from crude pyrolysis oil feedstocks. For example, the phenolic fraction contained compounds such as 4-isopropylphenol and 4-isopropyl-3-methylphenol that can be utilized to form for example, novolak resins. In addition, 4,4′-isopropylidenediphenol present in the phenolic fraction can be utilized as a glycidation substrate. The organic fraction (which corresponds to the second organic stream 175) contained 2-methyl-2-(4′-hydroxyphenyl) pentanone-4 and 4-isopropyl-3-methylphenol. Each of these compounds can be utilized to form for example, novolak resins. It is noted that the oily, viscous composition of the pyrolysis oil utilized caused accumulation of phenol in the oily oligomeric fraction.

In sum, the data shows that separation of organic streams from solvolysis and pyrolysis of an epoxy-resin composite material can be achieved utilizing embodiments of the present disclosure.

### Example 2: Infrared Spectroscopy

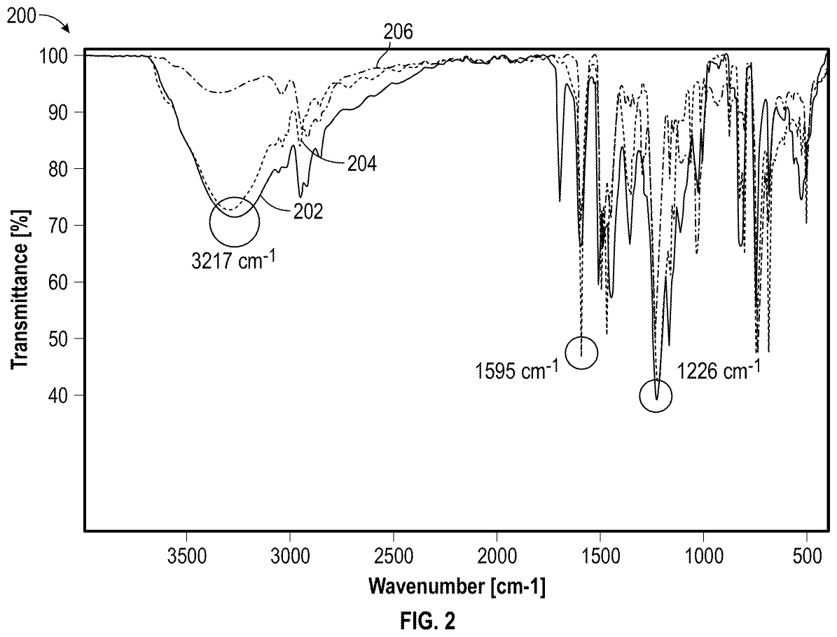

The composition of the oligomeric fraction, the phenolic fraction, and the organic fraction were investigated by infrared (IR) spectroscopy. FIG. 2 shows an overlay of IR spectra 200 of the oligomeric fraction 202, phenolic fraction 204, and the organic fraction 206 for the feedstock comprising pyrolysis oil. Various functional groups were detected, including a hydroxyl (O—H) stretch at about 3217 cm⁻¹, an aromatic C═C bend at about 1595 cm⁻¹, and a C—H stretch at about 1226 cm⁻¹. It is noted that phenols (C—H) typically appear as doublet signals around the latter region.

Overall, the IR spectra indicates that the organic fraction 206 is low in phenol, low in phenolic oligomers, and low in higher molecular weight phenol derivatives. In contrast, the characteristic O—H stretch at about 3217 cm⁻¹ and C—H stretch at about 1226 cm⁻¹ are clearly present in the oligomeric fraction 202 and the phenolic fraction 204.

### Example 3: Gel Permeation Chromatography

The composition of the organic fraction, the oligomeric fraction, the phenolic fraction, and for the separations of the pyrolysis oil and the solvolysis oil were investigated by gel permeation chromatography (GPC). GPC measurements provide an overview of the molecular weight distribution of the individual fractions.

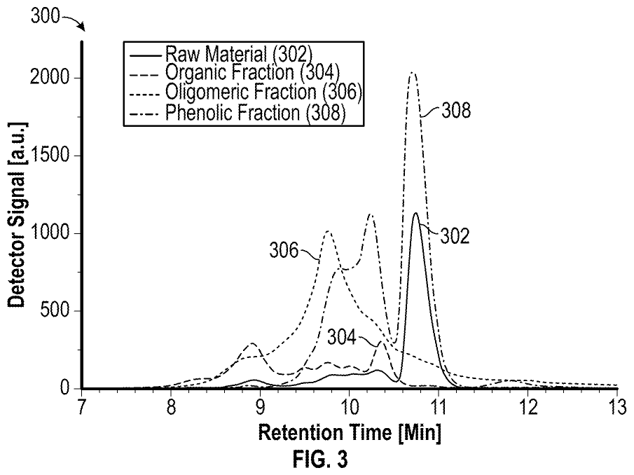

Analysis of fractions from separation of pyrolysis oil. FIG. 3 shows an overlay of GPC chromatograms 300 of crude pyrolysis oil (the raw material 302) and separated fractions (the organic fraction 304, the oligomeric fraction 306, and the phenolic fraction 308). Table 2 selected GPC data of the raw pyrolysis oil and fractions. In Table 2, Mₙ is the number-average molecular weight, M_{w} is the weight-average molecular weight, and M_{z} is the Z-average molecular weight. PDI refers to polydispersity index and is defined as the weight-average molecular weight (M_{w}) divided by number-average molecular weight (Mₙ), (M_{w}/Mₙ).

TABLE 2

| Sample | Mₙ, g/mol | M_{w}, g/mol | M_{z}, g/mol | PDI |
|---|---|---|---|---|
| Pyrolysis oil | 204 | 300 | — | 1.47 |
| (raw material) 302 | | | | |
| Organic fraction 304 | 233 | 350 | 680 | 1.50 |
| Oligomeric fraction 306 | 177 | 226 | 389 | 1.28 |
| Phenolic fraction 308 | 144 | 190 | 797 | 1.31 |

The data in Table 2 indicates that processes described herein can be utilized to separate valuable fractions from crude feedstocks. Here, the molecular weights of the individual fractions from pyrolysis oil indicate a clear separation of the oil into fractions of different sizes. The PDI of the oligomeric fraction and the phenolic fraction from the pyrolysis oil separation was determined to be close to 1 indicating that each of these fractions was close to homogeneous. Overall, the data for the pyrolysis oil fractions indicated that valuable fractions can be separated from the pyrolysis oil feedstock.

Analysis of fractions from separation of solvolysis oil. The composition of each of the organic fraction, the oligomeric fraction, the phenolic fraction separated from the solvolysis oil were also investigated by GPC. The M_{w} values of the individual fractions from the solvolysis oil separation were determined to be in the following order:

organic fraction >oligomeric fraction >raw material >phenolic fraction

After separation of the solvolysis oil, the organic fraction had the largest M_{w} value as compared to the phenolic fraction and the oligomeric fraction, and the phenolic fraction had the lowest M_{w} value. The oligomeric fraction had a larger M_{w} value than the phenolic fraction.

The Mₙ values of the individual fractions from the solvolysis oil separation were determined to be in the following order with the organic fraction having the highest Mₙ value and the phenolic fraction having the lowest Mₙ value:

organic fraction >oligomeric fraction >raw material >phenolic fraction

The M_{z} values of the individual fractions from the solvolysis oil separation were determined to be in the following order with the oligomeric fraction having the highest M_{z} value and the phenolic fraction having the lowest M_{z} value (the M_{z} of the raw material was not determined):

oligomeric fraction >organic fraction >phenolic fraction

The PDI values of the individual fractions from the solvolysis oil separation were determined to be in the following order with the raw material having the highest PDI value and the phenolic fraction having the lowest PDI value:

raw material >oligomeric fraction >organic fraction >phenolic fraction

The data for the solvolysis oil fractions indicated that valuable fractions can be separated from the solvolysis oil feedstock. The chromatograms and data from the GPC indicated that embodiments described herein can successfully separate higher molecular weight components in the oligomeric fraction and lower molecular weight components in the phenolic fraction. The PDI of the phenolic fraction from the solvolysis oil separation was determined to be close to 1 indicating that the phenolic fraction was close to homogeneous.

### Example 4: Novolak Resin Synthesis and Characterization

Formation of novolak resins by embodiments described herein were performed. The source of formaldehyde used for the syntheses of novolak resins was formalin. The formalin was a 45% formaldehyde solution in water. Oxalic acid was used as the catalyst for the syntheses.

The general reaction scheme for forming novolak resins is shown in Scheme 3, though it should be understood that phenolic compounds of formula (II) can also be utilized to form a novolak resin as described above in Scheme 2. In Scheme 3, A is phenol, B represents the source of formaldehyde, and C represents the novolak resin product. In some examples, n was generally determined to be from about 1 to about 5.

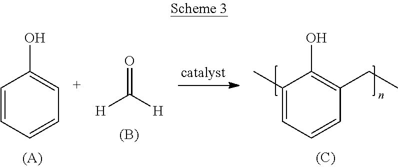

General Procedure for Synthesizing Novolak Resins. Phenol (about 72.18 g, about 0.767 mol, about 1.0 equivalent) was placed in a multi-neck flask, mixed, and heated to a temperature of about 95° C. Oxalic acid (about 0.345 g, about 0.038 mol, about 0.005 equivalent) in water (about 0.35 mL, about 0.005 equivalent) was then added to the flask. Formalin (about 25.59 g, about 0.383 mol, about 0.5 equivalent) was added slowly over a period of about 1 h under reflux and the reaction solution was then stirred for another 1 h under reflux to form a product mixture. Excess phenol was removed by distilling the product mixture at normal pressure, emptying the receiver, and then heating to a temperature of about 140° C. under vacuum

Comparative Novolak Resin. The general procedure was followed to form a comparative novolak resin (Ex. 406) using 100 wt % of pure phenol (commercial grade phenol).

Example Novolak Resins. The phenolic fractions (compositions enriched in phenol) from the separation process of the pyrolysis oil and the solvolysis oil were investigated for their use in the synthesis of novolak resins. The general procedure was followed for the synthesis of example novolak resins. In these examples, 20 wt % or 100 wt % of the pure phenol was replaced with a phenolic fraction.

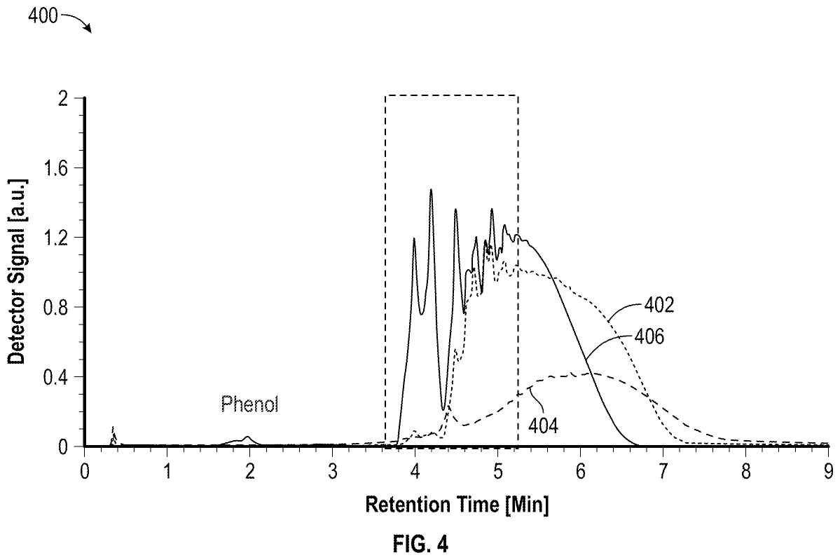

Novolak resins produced from the phenolic fraction of pyrolysis oil. FIG. 4 shows an overlay of UHPLC chromatograms 400 of novolak resins produced from 20% or 100% recycled material (phenolic fraction). Ex. 402 and Ex. 404 refer to the novolak resins made with 20% and 100 wt %, respectively, of the phenolic fraction from the separation of pyrolysis oil. Table 3 shows selected GPC data of the example novolak resins (Ex. 402, and Ex. 404), and the comparative novolak resin (Ex. 406). In Table 3, “n” refers to the repeating unit in Scheme 3 having a molecular weight of about 106 g/mol.

TABLE 3

| | Phenolic | Pure | | | | | |
|---|---|---|---|---|---|---|---|
| Ex. | fraction, | phenol, | Mₙ, | M_{w}, | M_{z}, | | |
| No. | wt % | wt % | g/mol | g/mol | g/mol | PDI | n |
| 402 | 20 | 80 | 211 | 271 | 281 | 1.28 | 1.9 |
| 404 | 100 | 0 | 449 | 738 | 485 | 1.64 | 4.2 |
| 406 | 0 | 100 | 206 | 272 | 264 | 1.32 | 1.9 |

When comparing the comparative novolak reference (Ex. 406) with the example novolak resins (Ex. 402, and Ex. 404), each of the example novolak resins showed typical molar mass distributions in the low molecular weight range. Overall, the data indicated that the phenolic fractions from the pyrolysis oil can be successfully converted to novolak resins at both 20% recycled phenolic content (Ex. 402) and 100 wt % recycled phenolic content (Ex. 404).

The UHPLC measurement of the various novolak resins shown in FIG. 4 indicated that novolak oligomers have been formed. This result is particularly evident in the region between 3.6 and 5.2 minutes (dashed box). This region shows a typical distribution of low molecular weight oligomers and the respective repeat unit “n” (n=1, 2, 3, 4, and 5). The different “n” of the samples corresponded to the repeat unit calculated by Mₙ of the molecular weight distribution and a repeat unit of 106 g/mol (Corresponds exactly to one unit in the polymer backbone).

The PDI values shown in Table 3 indicate the distribution of the polymer. For example, the broad distribution in the case of 100 wt % phenolic fraction from pyrolysis oil (Ex. 404; PDI of about 4.2) can indicate that several reactions have probably taken place or that the reaction has not stopped quickly or reacted thoroughly. The low PDI for the 20 wt % phenolic fraction from pyrolysis oil (Ex. 402; PDI of about 1.9) matched that of the comparative novolak reference (Ex. 406).

In addition, the 100 wt % phenolic fraction from pyrolysis oil (Ex. 404) likely showed a different distribution since it contains a high proportion of phenolic derivatives compared to all other compositions. This result indicated that the novolak resins formed from the 100 wt % phenolic fraction from pyrolysis oil had higher molecular weights, since the monomers used are larger than pure phenol (M_{w} of 94.11 g/mol). In addition, the phenol derivatives (for example, phenolic compounds and phenolic oligomers) can react differently than pure phenol during polymerization. Use of the phenolic fractions from the separation of pyrolysis oil (Examples 402 and 404) showed that even with a different composition of phenol derivatives (for example, phenolic compounds and phenolic oligomers), polycondensation with formaldehyde to form novolak resins is feasible.

Novolak resins produced from the phenolic fraction of solvolysis oil. The phenolic fraction (composition enriched in phenol) from the separation process of the solvolysis oil was also investigated for its use in the synthesis of novolak resins. The general procedure was followed for the synthesis of example novolak resins. In these examples, 20 wt % or 100 wt % of the pure phenol was replaced with a phenolic fraction from the solvolysis oil separation. The data indicated that the phenolic fractions from the solvolysis oil can be successfully converted to novolak resins at both 20% recycled phenolic content and 100 wt % recycled phenolic content.

In comparison to the comparative novolak reference (Ex. 406) each of the example novolak resins formed from the phenolic fraction of the solvolysis oil showed typical molar mass distributions in the low molecular weight range. Use of the phenolic fractions (content of phenol >80 wt %) from the separation of solvolysis oil can be utilized to form a product novolak resin having a low polydispersity and is thus comparable to the conventional synthesis of pure novolak resins from phenol and formaldehyde. Overall, the results indicated that the phenolic fractions from the solvolysis oil separation can be utilized for forming, for example, novolak resins.

Embodiments of the present disclosure generally relate to processes for separating organic products from organic recycle streams, and more specifically to processes for producing a composition enriched in phenol from organic recycle streams. Embodiments described herein can enable removal of phenol fractions and other organic fractions from resin recycle streams and resin waste streams. The phenol fractions and other organic fractions can be utilized to produce, for example, novolak resins and epoxy resins, among other materials.

As used herein, reference to an R group, alkyl, substituted alkyl, hydrocarbyl, or substituted hydrocarbyl without specifying a particular isomer (such as butyl) expressly discloses all isomers (such as n-butyl, iso-butyl, sec-butyl, and tert-butyl). For example, reference to an R group having 4 carbon atoms expressly discloses all isomers thereof. When a compound is described herein such that a particular isomer, enantiomer or diastereomer of the compound is not specified, for example, in a formula or in a chemical name, that description is intended to include each isomer and enantiomer of the compound described individual or in any combination.

As is apparent from the foregoing general description and the specific aspects, while forms of the aspects have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “Is” preceding the recitation of the composition, element, or elements and vice versa, such as the terms “comprising,” “consisting essentially of,” “consisting of” also include the product of the combinations of elements listed after the term.

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. For example, the recitation of the numerical range 1 to 5 includes the subranges 1 to 4, 1.5 to 4.5, 1 to 2, among other subranges. As another example, the recitation of the numerical ranges 1 to 5, such as 2 to 4, includes the subranges 1 to 4 and 2 to 5, among other subranges. Additionally, within a range includes every point or individual value between its end points even though not explicitly recited. For example, the recitation of the numerical range 1 to 5 includes the numbers 1, 1.5, 2, 2.75, 3, 3.80, 4, 5, among other numbers. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, aspects comprising “a phenolic compound” includes aspects comprising one, two, or more phenolic compounds, unless specified to the contrary or the context clearly indicates only one phenolic compound is included.

While the foregoing is directed to aspects of the present disclosure, other and further aspects of the disclosure may be devised without departing from the basic scope thereof, and the scope thereof is determined by the claims that follow.

What is claimed is:

1. A process, comprising:
(a) contacting a feedstock with a first organic solvent and water and forming a first aqueous stream comprising organic acids and a first organic stream comprising phenol, phenolic compounds, and phenolic oligomers, the feedstock having a first concentration of the phenol;
(b) contacting the first organic stream with an aqueous base and forming a second aqueous stream comprising the phenol and the phenolic oligomers and a second organic stream comprising the phenolic compounds;
(c) contacting the second aqueous stream with an aqueous acid and forming a precipitate comprising the phenolic oligomers and a third aqueous stream comprising the phenol; and
(d) contacting the third aqueous stream with a second organic solvent and forming a composition having a second concentration of the phenol that is greater than the first concentration of the phenol.

2. The process of claim 1, wherein the feedstock comprises products from solvolysis of an epoxy-resin composite material, pyrolysis of an epoxy-resin composite material, thermolysis of an epoxy-resin composite material, catalytic depolymerization of an epoxy-resin composite material, or combinations thereof.

3. The process of claim 1, wherein the second organic stream further comprises a glycidation substrate, a curing agent, an accelerator, or combinations thereof.

4. The process of claim 3, wherein the glycidation substrate comprises a bisphenol.

5. The process of claim 1, wherein the second organic stream comprises a lower concentration of the phenol than a concentration of the phenol in the second aqueous stream.

6. The process of claim 1, wherein the aqueous acid comprises an inorganic acid, an organic acid, or combinations thereof.

7. The process of claim 6, wherein the inorganic acid is selected from the group consisting of hydrochloric acid, sulfuric acid, nitric acid, phosphoric acid, and combinations thereof.

8. The process of claim 6, wherein the organic acid is selected from the group consisting of formic acid, acetic acid, citric acid, oxalic acid, and combinations thereof.

9. The process of claim 1, wherein the aqueous base comprises an alkali metal hydroxide.

10. The process of claim 9, wherein the alkali metal hydroxide is selected from the group consisting of lithium hydroxide, sodium hydroxide, potassium hydroxide, and combinations thereof.

11. The process of claim 1, wherein each of the first and second organic solvents, independently, comprise an aprotic solvent.

12. The process of claim 1, wherein each of the first and second organic solvents are independently selected from the group consisting of halogenated solvent, ether solvent, ketone solvent, ester solvent, and combinations thereof.

13. The process of claim 1, wherein each of the first and second organic solvents are independently selected from the group consisting of dichloromethane, chloroform, methyl tert-butyl ether, ethyl acetate, toluene, benzene, hexane, cyclohexane, and combinations thereof.

14. The process of claim 1, wherein operation (c) further comprises:
adding the aqueous acid to the second aqueous stream and forming a mixture having a pH of about 5 or less.

15. A process, comprising:
contacting a feedstock comprising thermochemically derived products of an epoxy-resin composite material, catalytically depolymerized products of an epoxy-resin composite material, or combinations thereof with an aprotic solvent and water and forming a first aqueous stream comprising organic acids and a first organic stream comprising phenol, phenolic compounds, and phenolic oligomers, the feedstock having a first concentration of the phenol; and
contacting the first organic stream with an aqueous base and forming a second aqueous stream comprising the phenol and the phenolic oligomers and a second organic stream comprising the phenolic compounds, wherein:
the second organic stream further comprises a glycidation substrate, a curing agent, an accelerator, or combinations thereof;
the aqueous base comprises an alkali metal hydroxide; and
the second organic stream comprises a lower concentration of the phenol than the second aqueous stream.

16. The process of claim 15, wherein the alkali metal hydroxide comprises lithium hydroxide, sodium hydroxide, potassium hydroxide, or combinations thereof.

17. The process of claim 15, wherein the thermochemically derived products of the epoxy-resin composite material comprise a solvolysis oil, a pyrolysis oil, or combinations thereof.

18. The process of claim 15, wherein the aprotic solvent is selected from the group consisting of dichloromethane, chloroform, methyl tert-butyl ether, ethyl acetate, and combinations thereof.

19. A process for forming a phenol-formaldehyde resin, the process comprising:
contacting a feedstock comprising products from solvolysis of an epoxy-resin composite material, pyrolysis of an epoxy-resin composite material, thermolysis of an epoxy-resin composite material, catalytic depolymerization of an epoxy-resin composite material, or combinations thereof with a first organic solvent and water and forming a first aqueous stream comprising organic acids and a first organic stream comprising phenol, phenolic compounds, and phenolic oligomers, the feedstock having a first concentration of the phenol;
contacting the first organic stream with an aqueous base and forming a second aqueous stream comprising the phenol and the phenolic oligomers and a second organic stream comprising the phenolic compounds;
contacting the second aqueous stream with an aqueous acid and forming a precipitate comprising the phenolic oligomers and a third aqueous stream comprising the phenol;
contacting the third aqueous stream with a second organic solvent and forming a composition having a second concentration of the phenol that is greater than the first concentration of the phenol; and
converting the composition enriched in the phenol to a phenol-formaldehyde resin.

20. The process of claim 19, wherein the converting the composition enriched in the phenol to the phenol-formaldehyde resin comprises:
forming a mixture comprising the composition enriched in the phenol, an aldehyde or ketone, and a catalyst; and
reacting the mixture and forming the phenol-formaldehyde resin.
